(19) United States
(12) Patent Application Publication
(10) Pub. No.: US 2018/0045675 A1
(43) Pub. Date: Feb. 15, 2018

(54) SINGLE-CELL INTRACELLULAR NANO-PH PROBES

(71) Applicant: The Regents of the University of California, Oakland, CA (US)

(72) Inventors: Rifat Emrah OZEL, San Jose, CA (US); Nader POURMAND, Scotts Valley, CA (US)

(21) Appl. No.: 15/552,685

(22) PCT Filed: Feb. 24, 2016
(86) PCT No.: PCT/US2016/019333

§ 371 Date: Aug. 22, 2017

Related U.S. Application Data
(60) Provisional application No. 62/120,624, filed on Feb. 25, 2015.

(51) Int. Cl. G01N 27/416; G01N 27/49; G01N 33/487; G01N 21/80

(52) U.S. Cl. CPC G01N 27/4167; G01N 21/80; G01N 27/49; G01N 33/48728

(57) ABSTRACT

Disclosed is a method and device for sensing pH in a single living cell. The device is constructed for directing a nano-sized probe to pierce a single cell and extract accurate pH measurements in real time therefrom. A nanopipette, containing an electrode, is prepared through physisorption of chitosan, a biocompatible pH-responsive polymer, onto highly hydroxylated quartz nanopipettes with extremely small pore size (−97 nm). Changes of pH alter the surface charge of chitosan, which can be measured as a change in ionic current at the nanopore. The dynamic pH range of the nano-pH probe was from 2.6 to 10.7 with a sensitivity of 0.09 pH units. The present device can be used for single-cell intracellular pH measurements using, for example, non-cancerous and cancerous human cells, including human fibroblasts and model cells such as HeLa (epithelial cervix).

### CROSS-REFERENCE TO RELATED APPLICATIONS

This application claims priority from U.S. Provisional Patent Application No. 62/120,624 filed on Feb. 25, 2015, which is hereby incorporated by reference in its entirety.

### STATEMENT OF GOVERNMENTAL SUPPORT

This invention was made with Government support under Contract Number U54CA143803 awarded by the National Cancer Institute, Contract Number P01-35HG000205 awarded by the National Institutes of Health, and Contract Number R21NS082927 awarded by the National Institute of Neurological Disorders and Stroke. The Government has certain rights in the invention.

### REFERENCE TO SEQUENCE LISTING, COMPUTER PROGRAM, OR COMPACT DISK

None.

### BACKGROUND OF THE INVENTION

### Field of the Invention

The present invention relates to the field of nanopore scale devices and sensors, in particular for pH sensing of fluids and solutions within a single cell.

### Related Art

Presented below is background information on certain aspects of the present invention as they may relate to technical features referred to in the detailed description, but not necessarily described in detail. That is, individual compositions or methods used in the present invention may be described in greater detail in the publications and patents discussed below, which may provide further guidance to those skilled in the art for making or using certain aspects of the present invention as claimed. The discussion below should not be construed as an admission as to the relevance or the prior art effect of the patents or publications described.

Personalized medicine holds great potential, especially in treating cancer, which remains a major medical challenge due to both intrinsic and acquired resistance to conventional chemotherapeutics¹⁻³. In the last decade, advances have been made in the development of personalized cancer therapeutics to increase the efficacy of chemotherapy⁴. Despite every effort to tailor drugs to the individual, results vary⁵. This fact has been correlated with the presence of genetically distinct cells within an individual tumor⁶. In recent studies genome sequencing technology has been employed to identify these genetic alterations in a large population of cells⁷⁻⁹. While genetic aspects of cancer cell heterogeneity and the relationship between mutations and drug resistance have been studied extensively, development of pre-screening technologies to detect heterogeneity, that is, to find cancer cells that differ in their cellular metabolism and physiology within large cell populations, is under-investigated.

Evaluation of cell heterogeneity can be performed through the measurement of cytoplasmic ions and molecules. Accumulation of metal ions¹⁰, changes in reactive oxygen (ROS) and nitrogen species (RNS) levels¹¹, and protein expression¹² are important markers of cancerous cells within cell populations. Although less recognized, pH is also a distinctive factor of cancer cells. pH is one of the most intriguing features in initiating and regulating a myriad of cellular events, such as multi-drug resistance in tumors¹³, protein processing¹⁴, endocytosis¹⁵ and apoptosis¹⁶. Due to its vital importance, the pH of the intracellular environment is strictly regulated through various ion channels and intracellular weak acids and bases, such as alkali cation-H+ exchangers, bicarbonate and acid loading transporters. In mammalian cells, subcellular compartments have different pH values in order to sustain optimum operational conditions for certain metabolic functions¹⁷. In normal physiological conditions, the resting intracellular pH of mammalian cells is maintained between 6.8 and 7.3¹⁸. On the other hand, extracellular pH values are slightly alkaline with the range of 7.2 to 7.4. A dysregulation of intracellular pH is often associated with altered cell functions, proliferation and drug resistance, and is observed in cancerous tumors¹⁹. Moreover pH has a great effect on tumor growth and cancer cell migration and therefore the potential for metastases^{20,21}.

Carcinogenic tumors are heterogeneous and widely assumed to be acidic due to the high metabolic rate of cancer cells coupled with poor blood supply. This regional high metabolism and lack of perfusion triggers an anaerobic metabolism which leads extracellular pH levels to decrease to ˜6.0²². Additionally, aerobic metabolism can increase the intracellular concentration of carbon dioxide (CO₂), which results in a decrease of local pH levels. These two mechanisms of acidosis are commonly accepted in cancer research. Little is known, however, about whether intracellular pH levels contribute to intratumoral heterogeneity, and if it is an indicator of preexisting metabolic heterogeneity in cancer cells in a large cell population. Greater granularity of pH data will be of great importance not only for the development of new anti-cancer drugs and carriers, as most new drug delivery systems propose to use pH sensitive polymers or pH sensitive polymeric nanoparticles, but also to ascertain how effectively anti-cancer drugs work over the course of treatment. Therefore, real-time quantitative measurement of intracellular pH may be crucial to link intratumoral cell heterogeneity, drug resistance and drug delivery systems for effective treatment.

pH can be used as a marker for the identification of variants of cancer cells in a tumor tissue. Once identified, these cells can be tagged and followed over the course of drug treatment. Then samples can be collected from the tagged cells to sequence their RNA and DNA to illuminate what makes these cells drug-resistant.

Detecting pH at the cellular level is not only important to investigate single cancer cells and cell heterogeneity in a tumor environment but also to understand neurodegeneration and aging. Neurodegenerative diseases, such as Parkinson's and Alzheimer's diseases, create heterogeneous physico-chemical environments due to mitochondrial oxidative phosphorylation, and therefore it is important to measure pH and understand its effect on neural recovery at the damaged site of brain²³. Additionally, cerebral pH has been found to be one of the major markers of metabolic disturbance and lethality after brain injury²⁴. Many of these studies have suffered from the lack of an appropriate analytical tool.

Commonly utilized analytical techniques to measure intracellular pH values include nuclear magnetic resonance (NMR)²⁵, electrochemistry^{26,27,} confocal microscopy²⁸, and absorbance and fluorescence spectroscopy^{29,31}. Of these, fluorescence spectroscopy and imaging are the most widely used techniques. However, fluorescence intensity is hard to quantify directly and suffers from experimental factors such as dye localization, photobleaching, excitation wavelength and cellular uptake and release rate. Additionally, fluorescence intensity can be affected by autofluorescence. Moreover, fluorescence probes do not allow continuous and site-specific detection of intracellular pH levels.

Thus, intracellular pH is both an indicator of cell metabolism and also plays an important role in the initiation and regulation of a myriad of cellular functions such as multi-drug resistance, protein processing and apoptosis. Even within a large clonal population, such as cancerous tumor entities, cells are not identical, and the differences of intracellular pH levels of individual cells may be important indicators of heterogeneity that could be relevant in clinical practice, especially as we move toward more personalized medicine. Therefore, the detection of intracellular pH at the single-cell level is of great importance to identify and study outlier cells. However, quantitative and real-time measurement of intracellular pH of individual cells within a cell population is challenging with existing technologies, and there is a need to engineer new methodologies.

### Specific Patents and Publications

Functionalized Nanopipette Biosensor, Karhanek et al. in US Patent Application Publication 2010/0072080, published on Mar. 25, 2010, disclose methods and devices for biomolecular detection, comprising a nanopipette, exemplified as a hollow inert, non-biological structure with a conical tip opening of nanoscale dimensions, suitable for holding an electrolyte solution which may contain an analyte such as a protein biomolecule to be detected as it is passed through the tip opening.

Nanopore Device for Reversible Ion and Molecule Sensing or Migration, Pourmand et al. in US Patent Application Publication 2012/0222958, published on Sep. 6, 2012, disclose methods and devices for detection of ion migration and binding, utilizing a nanopipette adapted for use in an electrochemical sensing circuit. Chitosan is used on a PAA (polyacrylic acid) layer attached first to the nanopipette, and for measuring binding of ions such as copper.

Actis et al. in “Functionalized nanopipettes: toward label-free, single cell biosensors,” Bioanalytical Reviews 1:177-185 (2010) disclose a nanopipette as a label-free biosensor capable of identifying DNA and proteins.

Umehara et al. in “Label-free biosensing with functionalized nanopipette probes,” Proc. Nat Acad. Sci. 106(12): 4611-4616 (2009) disclose a label-free, real-time protein assay using functionalized nanopipette electrodes. The proteins interact with the nanopipette tip coated with probe molecules. It is shown that electrostatic, biotin-streptavidin, and antibody-antigen interactions on the nanopipette tip surface affect ionic current flowing through a 50-nm pore.

### BRIEF SUMMARY OF THE INVENTION

The following brief summary is not intended to include all features and aspects of the present invention, nor does it imply that the invention must include all features and aspects discussed in this summary.

The present invention comprises, in certain embodiments, a device for measuring pH inside a single cell, comprising (a) a nanopipette structure that (i) is operatively connectable to a micromanipulator and sensing device for piercing a cell on a support, (ii) contains a working electrode therein, said (iii) contains a polymer coating that selectively absorbs hydrogen ions; (b) said nanopipette structure further connected to an amplifier circuit constructed to apply different voltages between the working electrode and a reference electrode in a solution and further constructed to measure an ionic current between the working electrode and the reference electrode under different voltages; and (c) logic means for correlating different ionic currents measured by said amplifier circuit with pH values within a cell outside the nanopipette structure.

In certain embodiments, the present invention comprises a device wherein the micromanipulator and sensing device comprises an SICM (scanning ion conductance microscope) and xyz controller controlling the nanopipette for movement to and into a single cell. In certain embodiments, the present invention comprises a device wherein the amplifying circuit comprises a detection circuit with gain controls and with a low pass filter for detecting ionic currents. In some embodiments, the present invention comprises a device comprising an array of nanopipette structures connected to a single logic means, as shown, e.g. in FIG. 16. In certain embodiments, the chitosan has a monomer number between about 30,000 and 60,000 units. The chitosan may comprise a hemeprotein attached thereto.

In some embodiments, the present invention comprises a device wherein the polymer coating is selected from the group consisting of sulfonated tetrafluorethylene copolymer (Nafion®), poly-1-lysine, and alginate. In certain embodiments, the present invention comprises a device wherein the amplifier circuit comprises a potentiostat connected to the reference electrode and responsive to input from an amplifier having an input from the working electrode. In further embodiments, the present invention comprises a device wherein the potentiostat is connected to a counter electrode that is also connected to the potentiostat's reference electrode. In certain embodiments, the present invention comprises a device wherein the working electrode and the counter electrode are Ag/AgCl.

In certain embodiments, the present invention comprises a device for measuring pH inside a single cell, comprising (a) a nanopipette electrically connected to a circuit that measures ionic current versus potential at various potentials and is attached to an insertion device for inserting the nanopipette into a single cell; (b) logic means for correlating a rectification value with known pH values, wherein a rectification value obtained in a cell can be correlated with a known rectification value, thereby providing an output identifying a measured pH value; (c) said nanopipette having a layer of chitosan material directly bound to the surface of the nanopipette and porous to hydrogen ions; and (d) a circuit comprising a reference electrode that also functions as an auxiliary electrode and is connected to a potentiostat.

In further embodiments, the present invention comprises a device wherein the logic means is programmed for scanning the potential of the working electrode at a given potential range with respect to the reference electrode by measuring the current at an auxiliary electrode. The device may comprise an i/V amplifier that is bridged by a filter selection and a sensitivity selection circuit, wherein the components are adjusted to adjust the detectable current range based on the current passing through the electrolyte solution.

In certain embodiments, the present invention comprises a method for making a device for measuring pH inside a single cell, comprising (a) preparing a nanopipette structure that (i) is operatively connectable to a micromanipulator and sensing device for piercing a cell on a support, (ii) contains a working electrode therein, and (iii) contains a polymer coating that selectively absorbs hydrogen ions; (b) connecting said nanopipette structure to an amplifier circuit constructed to apply different voltages between the working electrode and a reference electrode in a solution and further constructed to measure an ionic current between the working electrode and the reference electrode under different voltages; (c) connecting said nanopipette structure to logic means for correlating different ionic currents measured by said amplifier circuit with pH values within a cell outside the nanopipette structure.

In further embodiments, the present invention comprises a method as described above wherein said polymer coating is applied by binding a chitosan material layer to the nanopipette; further comprising connecting said working electrode to an amplifier that conducts and measures an I-V curve for ionic current through the nanopipette.

In certain embodiments, the present invention comprises a method of measuring pH in a cell, comprising (a) providing a nanopipette structure, having an interior layer responsive to pH ions, and being electrically connected by a working electrode to a circuit comprising a potentiostat configured to measure ionic current through said nanopipette structure versus potential at various potentials in a electrochemical cell containing said nanopipette structure and a reference electrode; (b) inserting said nanopipette structure into a cell in said electrochemical cell; and (c) using said circuit to measure said ionic current, wherein said current is correlated to a known pH.

In some embodiments, the present invention comprises a method as described above wherein said inserting said nanopipette comprises using an SICM and an x-y-z controller. In certain embodiments, the present invention comprises a method wherein said circuit further comprises an amplifying circuit comprising a detection circuit with gain controls and with a low pass filter for detecting ionic currents. In certain aspects and embodiments, the present invention comprises a method wherein said interior layer comprises a layer of chitosan material having an average pore size between 50 nm and 150 nm diameter. The chitosan may have a monomer number between about 30,000 and 60,000 units, and may comprise a hemeprotein attached thereto.

In certain embodiments, the present invention comprises a method as described above wherein the interior layer comprises a polymer coating that is selected from the group consisting of sulfonated tetrafluorethylene copolymer (Nafion®), poly-1-lysine, and alginate.

In certain embodiments, the present invention comprises a method as described above wherein the circuit comprises a potentiostat connected to the reference electrode and responsive to input from an amplifier in turn having an input from the working electrode. In further embodiments, the present invention comprises a method wherein the potentiostat is connected to a counter electrode connected to the reference electrode. The working electrode and the counter electrode may be Ag/AgCl.

In some embodiments, the present invention comprises a method as described above wherein the voltage is between 0.5V and 0.7V. In futher embodiments, the present invention comprises a method wherein a variety of voltages is set on the potentiostat.

In certain embodiments, the present invention comprises a method as described above wherein the pH value is taken on a cancerous cell and compared to a pH on a noncancerous cell.

### BRIEF DESCRIPTION OF THE DRAWINGS

FIGS. 1A, 1B, 1C and 1D consists of a graph and scanning electron micrographs showing properties of nanopipettes of the present invention. The graph in FIG. 1A is a comparison of ionic current rectifications of a bare and chitosan-modified quartz nanopipette. Both measurements were carried out with quartz nanopipettes filled with 10 mM PBS (pH 7.0). Without the chitosan material, the current scales linearly with the potential vs. Ag/AgCl. FIG. 1B is a scanning electron micrograph demonstrating a typical nanopipette pore opening. Also shown are SEM images of focused ion beam cut (FIG. 1C) bare nanopipette tip and (FIG. 1D) chitosan-modified nanopipette showing the chitosan layer on the inner surface of the nanopipette.

FIG. 2A-2B is a pair of scanning electron micrographs showing (FIG. 2A) the side view of a nanopipette tip, and (FIG. 2B) the pore of a chitosan-modified nanopipette.

FIG. 3A-3B consists of (FIG. 3A) a schematic illustrating reversible changes in surface charge of a nanopipette of the present invention as a result of pH and (FIG. 3B) a graph showing calibration of chitosan-modified nanopipettes within the physiologically relevant pH range from 6.02 to 8.04. All data points are represented as relative rectification ratios at +/−0.5 V vs. Ag/AgCl reference electrode. The error bars represent standard deviations for n=4 replicate measurements. 0.1 M PBS was used as supporting electrolyte. As can be seen, in an acidic condition, the chitosan layer will change from a negative surface to a mixture of negative and positive ions. A pH decrease will cause protonation of the polymer and the change in surface charge will cause current rectification that is detected in the present circuit.

FIG. 4A, 4B, 4C is a set of graphs showing (FIG. 4A) typical linear sweep voltammograms for acid titration of a chitosan-modified nanopipette and (FIG. 4B) typical linear sweep voltammograms for base titration of a chitosan-modified nanopipette. The graph in (FIG. 4C) is the corresponding calibration nano-pH probe between 2.59 and 10.83. The traces are in color in the original. In FIG. 4A, the lowest pH measured, 6.96, is shown with the arrow. The lower pH values show higher current at the −0.5 point shown. In FIG. 4B, the highest pH, 10.83, is shown with the arrow.

FIG. 5 is a graph showing the pH response of a bare nanopipette. The error bars indicate standard deviation for n=3 replicate measurements.

FIG. 6A-6B is a pair of graphs showing calibration of chitosan-modified nanopipettes in cell culture media. The medium in FIG. 6A is 1×MEM, and the medium in FIG. 6B is DMEM. Current responses were measured at a fixed bias potential of 0.6 V. The error bars represent standard deviations for n=4 replicate measurements.

FIG. 7A-7B is a pair of graphs showing current-potential curves of a chitosan-modified nanopipette for acid titration in cell culture media (FIG. 7A) MEM and (FIG. 7B) DMEM. The higher pH values are shown by arrows.

FIG. 8A-8B is a current trace obtained with a chitosan-modified nanopipette and a micrograph of the nanopipette. FIG. 8A shows the customized scanning ion conductance microscopy current-feedback signal recorded before, during and after cell penetration using an Axopatch 200B amplifier. Amplitude at y-axis is nanoamperes. FIG. 8B is the corresponding micrograph of the inserted chitosan-modified nanopipette.

FIG. 9A, 9B, 9C, 9D is a set of graphs showing intracellular pH levels of individual cells determined by chitosan-modified nanopipettes. pH levels were recorded for (FIG. 9A) human fibroblast, (FIG. 9B) HeLa, (FIG. 9C) MCF-7 and (FIG. 9D) MDA-MB-231 cells. Horizontal lines represent the average intracellular pH measured with the nano-pH probe.

FIG. 10A, 10B, 10C, 10D is a set of graphs showing representative current-potential curves of intracellular pH measurements with the chitosan-modified nanopipette for different cell types: (FIG. 10A) human fibroblast, (FIG. 10B) HeLa, (FIG. 10C) MCF7 and (FIG. 10D) MDA-MB-231. All readings for each type of cell line were obtained with a single pH nanoprobe. Cell 1 is shown by an arrow in (FIG. 10A), (FIG. 10C) and (FIG. 10D).

FIG. 11A, 11B, 11C consists of representative micrographs showing nano-pH probe insertion and a graph of current-voltage curves obtained with the nano-pH probe. The micrographs show (FIG. 11A) a nano-pH probe inserted into a MDA-MB-231 cell and (FIG. 11B) the insertion point after retraction of the probe. Cells did not show any morphological changes and stayed intact over the course of insertion and measurement, and survived after retraction. (FIG. 11C) Linear sweep voltammograms of regenerating baseline of nano-pH probe after cell interrogation in 0.1 M PBS (pH 7.0).

FIG. 12 is a graph showing real-time intracellular pH measurements with nano-pH probes. The pH measurements were performed on MDA-MB-231 cells in the absence (diamonds) and presence (cubes) of 100 μM NPPB (Cl⁻ channel blocker). The arrow in the figure shows the addition time of NPPB. Readings are obtained every 21 sec for 7 min post channel blocker exposure. Error bars represent standard deviation for n=3 replicates.

FIG. 13 is a graph representing pH changes over time of three MDA-MB-231 cells as a result of 100 μM NPPB (Cl⁻ channel blocker) exposure. Readings were obtained every 21 sec post channel blocker exposure.

FIG. 14A-14B shows (FIG. 14A) a diagrammatic representation of the present device wherein the nano-pH probe comprises a chitosan material layer. FIG. 14B shows the change in pH where an acidic condition causes an increased presence of protons on the polymer layer (top panel); it also shows rectification ratios (R_{pH}/Rₙₑᵤₜᵣₐₗ) increasing over a pH range of 6 (˜0.7) to 8 (˜1.1) (bottom panel).

FIG. 15 is a diagrammatic figure of the present circuitry that further clarifies the arrangement shown in FIG. 14A.

FIG. 16 is a schematic diagram showing a 2D sectional view of a nanoprobe array. Nanoprobes, each comprising a nanopipette containing a conductive material and connected to a working electrode, are mounted on an array. Each working electrode is connected, outside of the nanopipette, to a signal amplifier which has an input from both the working electrode and a common reference electrode.

### DETAILED DESCRIPTION OF THE PREFERRED EMBODIMENT

### Definitions

Unless defined otherwise, all technical and scientific terms used herein have the same meaning as commonly understood by those of ordinary skill in the art to which this invention belongs. Although any methods and materials similar or equivalent to those described herein can be used in the practice or testing of the present invention, the preferred methods and materials are described. Generally, nomenclatures utilized in connection with, and techniques of, cell and molecular biology and chemistry are those well-known and commonly used in the art. Certain experimental techniques, not specifically defined, are generally performed according to conventional methods well known in the art and as described in various general and more specific references that are cited and discussed throughout the present specification. For purposes of clarity, the following terms are defined below.

Ranges: For conciseness, any range set forth is intended to include any sub-range within the stated range, unless otherwise stated. As a non-limiting example, a range of 120 to 250 is intended to include a range of 120-121, 120-130, 200-225, 121-250 etc. The term “about” has its ordinary meaning of approximately and may be determined in context by experimental variability. In case of doubt, the term “about” means plus or minus 5% of a stated numerical value.

The term “nanopipette” means a hollow self-supporting, inert, non-biological structure with a conical tip opening of nanoscale, i.e., a nanopore, having a tip opening of 0.05 nm to about 500 nm, preferably about (+ or −20%) 50 nm or about 80 nm or about 100 nm. The hollow structure may be e.g. glass or quartz, and is suitable for holding inside of it a fluid which is passed through the tip opening. The interior of the nanopipette is selected or modified to minimize nonspecific binding of analyte. The interior of a nanopipette typically is in the form of an elongated cone, with a uniform wall thickness of a single layer of quartz or other biologically inert material, and is sized to allow insertion of an electrode that contacts solution in the nanopipette. The nanopipettes used herein typically have a single bore, but nanopipettes with multiple concentric bores can be prepared by pulling dual bore capillary tubes. The outer diameter is typically less than about 1 μm in the tip region.

The term “nanopore” means a small hole in an electrically insulating membrane, preferably the tip of a nanopipette, as described. The nanopore will be in a tip region, which is the last few mm of the nanopipette bore, adjacent the nanopore. The nanopore, as described below, is sized so that small molecular complexes will affect movement of ions and molecules through the nanopore. The nanopore is designed to function in a device that monitors an ionic current passing through the nanopore as a voltage is applied across the membrane. The nanopore will have a channel region formed by the nanopipette body, and, preferably, will be of a tapered, e.g. frusto-conical configuration. By pulling a quartz capillary as described below, a reproducible and defined nanopore shape may be obtained.

As described below, the term “nano-pH probe” refers to a device comprising a nanopipette containing an electrode inside and a functionalized interior portion, further comprising circuitry connected to the electrode to sense small changes in ionic current in the nanopipette, indicative of a pH in a material.

The term “quartz” means a nanopipette media is a fused silica or amorphous quartz, which is less expensive than crystalline quartz. Crystalline quartz may, however, be utilized. Ceramics and glass ceramics and borosilicate glasses may also be utilized but accuracy is not as good as quartz. The term “quartz” is intended and defined to encompass that special material as well as applicable ceramics, glass ceramics or borosilicate glasses. It should be noted that various types of glass or quartz may be used in the present nanopipette fabrication. A primary consideration is the ability of the material to be drawn to a narrow diameter opening. The preferred nanopipette material consists essentially of silicon dioxide, as included in the form of various types of glass and quartz. Fused quartz and fused silica are types of glass containing primarily silica in amorphous (non-crystalline) form.

The term “chitosan” is used herein in its conventional sense, to refer to a linear polysaccharide composed of randomly distributed β-(1-4)-linked D-glucosamine (deacetylated unit) and N-acetyl-D-glucosamine (acetylated unit). The amino group in chitosan has a pKa value of ˜6.5, which leads to a protonation in acidic to neutral solution with a charge density dependent on pH and the % DD (degree of deacetylation) value. This makes chitosan water soluble and a bioadhesive which readily binds to negatively charged surfaces such as mucosal membranes. Chitosan enhances the transport of polar drugs across epithelial surfaces, and is biocompatible and biodegradable. Chitosan is produced commercially by deacetylation of chitin, which is the structural element in the exoskeleton of crustaceans (crabs, shrimp, etc.) and cell walls of fungi. The degree of deacetylation (% DD) can be determined by NMR spectroscopy, and the % DD in commercial chitosans is in the range of 60-100%. On average, the molecular weight of commercially produced chitosan is between 3800 to 20,000 daltons.

The term “chitosan material” means the naturally occurring chitosan polysaccharide described above, and various allotropes and derivatives, described, e.g. in Rinaudo, “Chitin and chitosan: Properties and application,” Prog. Polym. Sci 31:603-632 (2006). As described there, chitosan can have a variety of degrees of solubility, acetylation or molecular weight. As described, the chitosan material or native chitosan may be formed in a layer that is thin and dilute so as to result in nanoscopic or microscopic pores that receive ions within the layer.

The term “highly hydroxylated” is used in connection with quartz materials (SiO₂) used in the present nanopore bearing hydroxyl groups. For example, α-quartz (0001) can be hydroxylated as described in Yang et al. “Water adsorption on hydroxylated α-quartz (0001) surfaces,” Phys. Rev. b 73:035406 (2006). See e.g. Konecny et al. “Reactivity of free radicals on hydroxylated quartz surface and its implications for pathogenicity experimental and quantum mechanical study,” J. Environ Pathol Toxicol Oncol. 2001; 20 Suppl 1:119-32.

The term “hemeprotein” refers to a metalloprotein containing a heme prosthetic group—an organic compound that allows a protein to carry out several functions that it cannot do alone. The heme contains a reduced iron atom, Fe2+ in the center of a highly hydrophobic, planar, porphyrin ring. Hemeproteins include hemoglobin, myoglobin, neuroglobin, cytoglobin and leghemoglobin.

The term pH has the commonly accepted definition, i.e., a measure of acidity or alkalinity of water soluble substances (pH stands for ‘potential of Hydrogen’). A pH value is a number from 1 to 14, with 7 as the middle (neutral) point. Values below 7 indicate acidity which increases as the number decreases, 1 being the most acidic. Values above 7 indicate alkalinity, which increases as the number increases, 14 being the most alkaline. This scale, however, is not a linear scale like a centimeter or inch scale (in which two adjacent values have the same difference).

The term “logic means” means a logical circuit that is programmable or is programmed to convert a series of electronic signals to one or more tangible measurable value(s). For example, U.S. Pat. No. 4,124,899 to Birkner, et al shows a programmable logic circuit which is referred to as a programmable array logic, or PAL, circuit. The present logic means produces a pH value based on a given change in ionic current though the described probe (containing a nanopipette sensitive and responsive to hydrogen ions and containing an electrode) relative to a reference probe. As will be appreciated, any required computer program may be loaded onto a computer, including without limitation a general purpose computer or special purpose computer, or other programmable processing apparatus to produce a machine, such that the computer program instructions which execute on the computer or other programmable processing apparatus create means for implementing the functions. Accordingly, appropriate logic means as used here may be software provided for use by a user on an extrinsic computer programmed to sense and control the present device.

### Overview of the Invention and Embodiments

The present invention provides a means and device that can measure pH within a single cell, as well as changes in pH in the cell, without the necessity of any exogenous materials. The measurement is in real time, and can track changes in pH while the nanopipette is inserted into the cell and the sensitive circuit measures ionic current at the nanopore opening of the nanopipette, which is in the cell, e.g. in the cytoplasm, nucleus, mitochondrion, etc. The detection circuit provides a high degree of sensitivity on the order of 0.1-0.01 pH units, with a described example showing detection of 0.09 pH units. Furthermore, the system has a large dynamic range, between about pH 2-11.

The present invention further comprises a method and device for measuring a current that varies in response to pH changes in a solution in a cell. In one method, the device is calibrated using different standard pH solutions. A calibration curve reflects current vs. pH and is calculated and used to measure pH in the sample. A preferred voltage setting for a current measurement is 0.6V, or within a range of 0.5V-0.7V. The measured current (measured with the potentiostat) increases as the pH in the sample decreases. The potentiostat reports the current and is swept across a voltage range to determine various responses and/or to determine an optimal operating voltage. Typically the applied voltage is swept from about 0.2 to 0.6V. In a presently preferred method of use, the potentiostat applies a chosen voltage to the system and records the current, that is correlated to the pH.

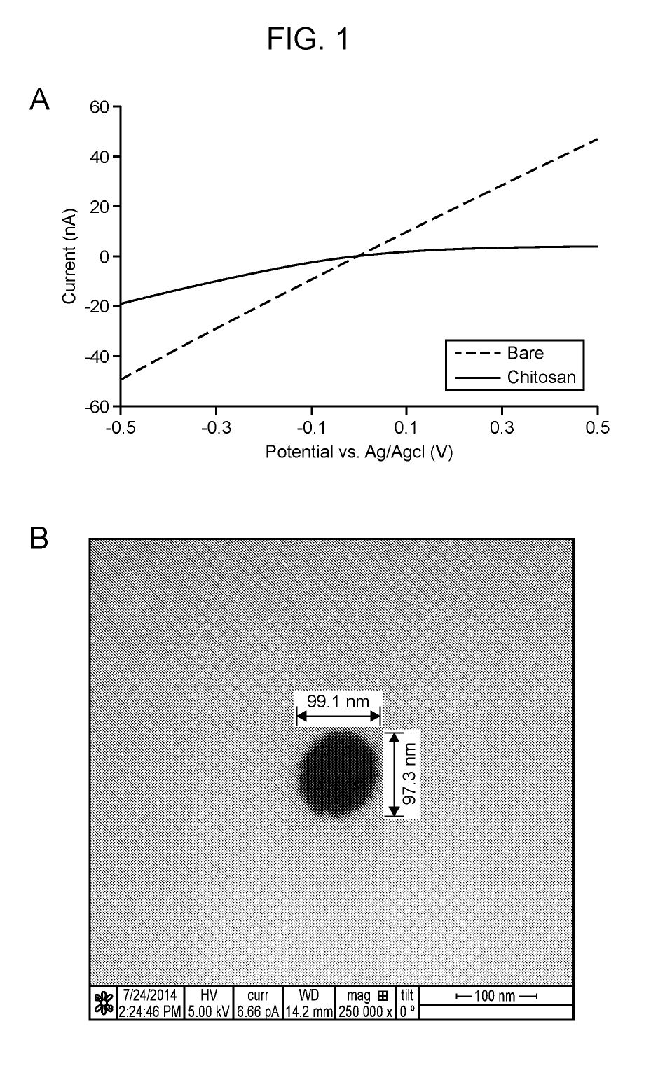

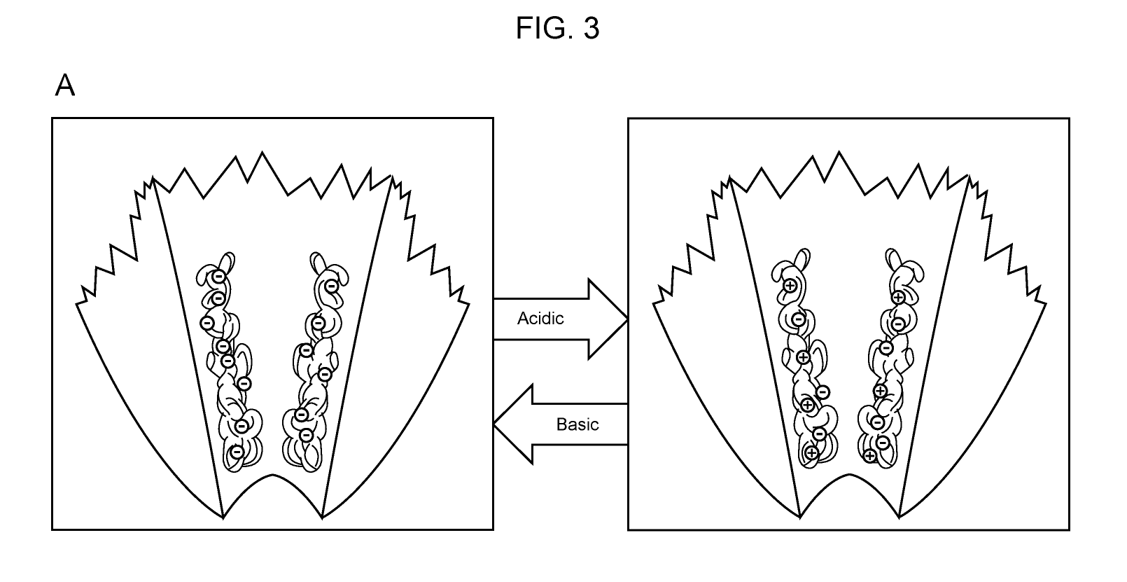

In one aspect, the invention comprises the use of a controlled concentration of highly porous chitosan material that forms a molecular sponge to trap ions including H+ to increase ionic rectification, as shown in the traces of FIG. 1A, FIG. 4A, 4B, etc. The highly porous coating (pore size approximately 50-150 nm, an average mean diameter of approximately 100 nm) permits a direct interaction with hydrogen ions present in the nano-pH probe. The permeable hydrogen ions (protons) generally have an ionic radius of about 0.012 Angstroms. The average pore size may be determined by microscopic means or calculated from a porosity value. See, e.g. Zeng et al., “Control of Pore Sizes in Macroporous Chitosan and Chitin Membranes,” Ind. Eng. Chem. Res., 1996, 35 (11), pp 4169-4175.

Ionic current rectification, as is known in the art, is characterized by an increase of the ion conduction for one voltage polarity but a decrease of it for the same voltage magnitude with opposite polarity, producing an asymmetric I-V curve. A positive and negative voltage is applied to the electrodes; the difference between the ionic current response is indicative of the pH in the pore, and, as a result, in the cell.

The highly porous chitosan material may be prepared by using a relatively low concentration of chitosan material in coating the nanopipette interior pore. In some aspects, the chitosan material is applied in a concentration of between 0.25% to 1% chitosan material. The chitosan material is directly bonded to hydroxyl groups on the quartz material of the nanopipette, in the vicinity of the interior of the nanopore. Preferably, short chain chitosan material is used, having a monomer number of about 30,000 to 60,000. Bonding may be enhanced by reacting the quartz with chemicals to increase surface functionality, such as sulfuric acid, hydrogen hydroxide, ammonium hydroxide, etc. This will serve to reduce contaminants and hydroxylate the quartz.

In another aspect, the present invention comprises modification of the chitosan material layer so as to contain a material sensitive to the redox potential in the cell. The redox potential of a cell is used in the conventional sense, to refer to a measure used to infer the direction and free energy cost of reactions involving electron transfer. The redox potential, or more accurately the reduction potential, of a compound refers to its tendency to acquire electrons and thereby to be reduced.

For example, one may use the redox potential to connect these two molecular protagonists, and estimate an upper bound on the number of ATP molecules that can be produced from the oxidation of NADH (produced for example in the TCA cycle). The redox potential of a cell may be perturbed by various diseases.

In another aspect, the present invention comprises a sensitive electronic device and arrangement of the working and reference electrodes used between the bulk solution and the interior of the nanopipette. The reference electrode also functions as an auxiliary electrode and is connected to a potentiostat. The system functions by scanning the potential of the working electrode at a given potential range with respect to the reference electrode by measuring the current at an auxiliary electrode. An i/V amplifier is bridged by a filter selection and a sensitivity selection circuit. These are used to adjust the detectable current range based on the current passing through the electrolyte solution.

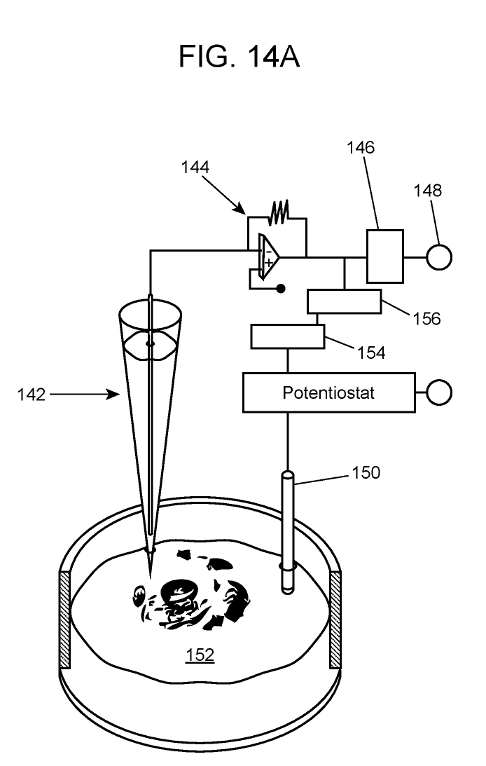

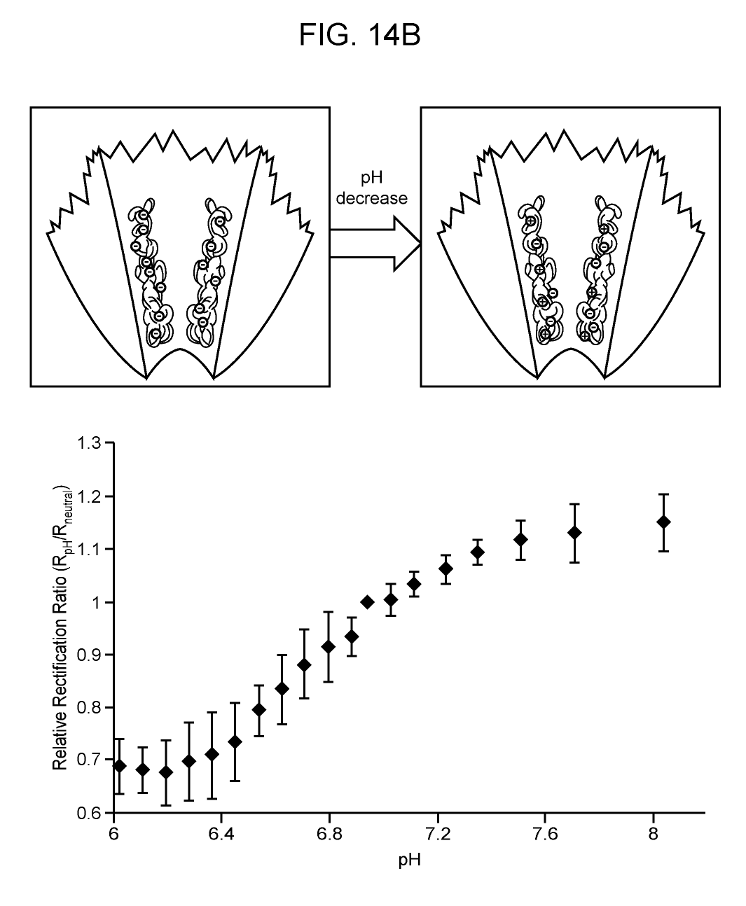

Referring now to FIG. 14A, the present device comprises a nanopipette 142 with a pH responsive polymer (e.g. chitosan) inside. The chitosan (responsive polymer, FIG. 14B) is directly adsorbed onto an internal surface of the nanopipette. The nanopipette contains a small opening structured to sense liquid in a cell injected by the nanopipette (opening less than about 200 nm, preferably between 10 and 20 nm). The nanopipette 142 is comprised in a system that also contains a reference electrode 150 (shown also in FIG. 15). The reference electrode 150 is connected to the input of a potentiostat which is further connected to a low pass filter 146 and from there to output 148. As described below, the working electrode is also connected to a potentiostat that injects current into the electrochemical cell 152 through a reference electrode. The working solution in the electrochemical cell also contains a reference electrode 150 connected to a potentiostat and an external electrode (not shown in FIG. 14A). The nanopipette 142 is inserted into a cell in a working solution (media) in the electrochemical cell, in which the reference electrode 150 is immersed.

As described below, the nanopipette (nano-pH probe) is operatively connected to a micromanipulator (not shown) such as a scanning ion conductance microscope that detects current feedback for positioning the nanopiette and inserting it into a selected cell.

As shown schematically in FIG. 14B, a pH decrease in the cell results in a protonation of the chitosan or equivalent polymer that can withdraw protons from the solution as it contacts the coating in the nanopipette 142. The change in the surface of the chitosan layer in the nanopore region affects the ionic current that can pass through the pore. The change in ionic current alters the output from the feedback amplifier shown generally at 144. The output is filtered by the low-pass filter 146 and is output at 148 to a monitor as described in connection with FIG. 15. The potentiostat is further connected to a gain selector 154 a digital attenuator 156.

FIG. 15 shows how the potentiostat as arranged in the present device achieves a high sensitivity of pH measurement within the cell. The nanopipette (142 shown in FIG. 14A) contains a working electrode within electrochemical cell 152 and shown as a hexagon. The electrochemical cell 152 is the solution that contains the single cell referred to above, and a conductive solution connecting the working electrode and the reference electrode 150. The reference electrode 150 also functions as an auxiliary electrode or counter electrode and is also connected to a potentiostat. As is shown and known (See U.S. Pat. No. 5,466,356 for details), the potentiostat provides hardware to operate in the electrochemical cell. The working electrode (in the nanopipette) is the electrode where the potential is controlled and where the current is measured. The reference electrode is used to measure the working electrode potential. A reference electrode should have a constant electrochemical potential as long as no current flows through it. The counter electrode completes the circuit with the working electrode. When the electrochemical environment is not very conductive (less than 1 uA), both reference and counter electrode can be attached to the same electrode.

The two circuits, shown in FIG. 15, operate simultaneously: A potential difference between reference electrode and working electrode is measured to identify the voltage in the electrochemical cell; and current is measured between working electrode and counter electrode. The current measurement between the working and the counter electrode will sense changes in pH. When the current is very small and when the electrode material is Ag/AgCl both counter and reference can work on a single electrode because the two simultaneous events can happen without any interference.

The system functions by scanning the potential of the working electrode at a given potential range with respect to the reference electrode by measuring the current at an auxiliary electrode. The potentiostat is connected to a gain selector 154 used to control the frequency at which the signal amplification is done. The working electrode (in the nanopipette) is connected to the input of an i/V (current to voltage) amplifier 158 that outputs to a digital attenuator 156 and from there back—as described above—to the reference electrode to create a feedback circuit. The i/V amplifier 158 further is bridged by a filter selection 162 and a sensitivity selection circuit 164. These are used to adjust the detectable current range based on the current passing through the electrolyte solution.

The amplifier 158 outputs to a low pass filter 146 and the output connection 148 (circle) shown connected to the low pass filter. This and the potentiostat provide input terminals (circles) to a monitor that can measure and record ionic current through the nanopipette. The monitor may comprise a computer programmed to monitor and control signals produced by the above components.

The computer will contain logic means that will convert a detected current, from the potentiostat circuit, to a pH value, based on a calibration established during use, or, alternatively built into the device.

The single cell in which the nanopipette is inserted may be a cell in culture in liquid or immobilized on a substrate. The single cell may be part of a tissue. It is identified microscopically and the nanopipette is controlled by an x-y-z controller to be inserted into the cell. Scanning ion-conductance microscopy (SICM) may be used for this purpose.

The present nano-pH probe can be used as an analytical tool to illuminate the relationship between pH and a variety of diseases. The present nano-pH probe may utilize scanning ion conductance microscopy (SICM) principles³². Nanopipettes are electrical devices that can measure the differences in ionic current at a nanopore. Their small size enables direct, real-time in vitro measurements with high spatial resolution and reduced invasiveness, allowing the monitoring of intracellular changes of an individual cell over the course of drug treatment. Recently nanopipettes have gained importance as novel sensing tools and have been investigated for the detection of proteins^{33,34}, metal cations^{32,35}, DNA³⁶ and carbohydrates³⁷. Quartz nanopipettes can be functionalized with various recognition materials. In this work, chitosan material, a biopolymer, is used as a pH-sensitive surface coating of the internal surface of nanopipettes. Chitosan is biocompatible and has low-toxicity which makes it ideal for biological purposes. It possesses unique film-forming ability, high adherence to surfaces and remarkable mechanical strength. In addition, chitosan has been shown as a selective coating for biosensor fabrication³⁸⁻⁴⁰.

It is demonstrated here the development and characterization of chitosan-modified quartz nanopipettes for pH measurements in physiological buffers and cell media. The chitosan-modified nanopipettes were then used for the direct measurement of intracellular pH in four different cells types, including human fibroblast, HeLa, MCF-7 and MDA-MB-231. As described, in vitro specificity of chitosan-modified nano-pH probes using a chloride channel blocker can be achieved. The nano-pH probe is a powerful candidate not only to investigate cell heterogeneity in a variety of pathologic states, including cancerous tumors, but also neurodegenerative states and aging.

The present device has been shown to overcome the limitations of intracellular pH measurement at the single-cell level. Direct measurement of intracellular pH has been demonstrated in a new way via simple physisorption of a chitosan material into a quartz nanopipette. This approach takes advantage of a pH-responsive chitosan polymeric layer and the small size of a nanopipette for intracellular pH measurement at the single-cell level. Described here is nano-pH probe prepared through physisorption of chitosan, a biocompatible pH-responsive polymer, onto highly hydroxylated quartz nanopipettes with extremely small pore size (˜97 nm). Changes of pH alter the surface charge of chitosan which can be measured as a change in ionic current at the nanopore. The dynamic pH range of the nano-pH probe was from 2.6 to 10.7 with a sensitivity of 0.09 pH units. Leveraging a scanning ion conductance microscope customized for single-cell navigation, we were able to insert nano-pH probes into individual cells. We have performed single-cell intracellular pH measurements using non-cancerous and cancerous cell lines, including human fibroblasts, HeLa, MDA-MB-231 and MCF-7, with the nano-pH probe. In vitro results showed that chitosan-functionalized nanopipettes measure intracellular pH selectively with high temporal resolution. The average intracellular pH levels were 7.37±0.29, 6.75±0.27, 6.91±0.20 and 6.85±0.11 for human fibroblast, HeLa, MCF-7 and MDA-MB-231, respectively. These results show good separation between fibroblast and cancerous cells, which have a more acidic cytoplasmic environment than non-cancerous cells. Additionally, our findings reveal that individual cells within a population may differ in their intracellular pH. We have further demonstrated the real-time continuous single-cell pH measurement capability of the sensor, showing cellular pH response to pharmaceutical manipulations. An NPPB exposure experiment demonstrates that the nano-pH probe enables real-time, continuous interrogation of a single cell upon biochemically induced changes in intracellular pH.

Our data show that chitosan-modified nanopipette sensing technology is a powerful approach for interrogating single-cell pH levels with high spatial and temporal resolution with high selectivity and sensitivity. Further application of this nano-pH probe technology may provide a deeper understanding of cell heterogeneity and drug resistance. To achieve this aim, we are working on the development of a fully automated system for high-throughput screening of cell populations over the course of drug treatment. Additionally, we will use nano-pH probes to investigate pH changes and differences in tumorous microenvironments (e.g. tumor tissues).

General Method and Materials

Reagents and materials. Chitosan (low molecular weight), 5-nitro-2-(3-phenylpropylamino)-benzoate (NPPB), sodium phosphate dibasic and monobasic were purchased from Sigma Aldrich. Sodium chloride (ACS grade), hydrochloric acid, sodium hydroxide and hydrogen peroxide were obtained from Fisher Scientific. Acetic acid (glacial) was supplied from Riedel-de-Haen. 2-propanol was obtained from Spectrum Chemicals. 2′,7′-bis-(2-carboxyethyl)-5-(and-6)-carboxyfluorescein acetoxymethyl ester (BCECF-AM) was bought from Invitrogen. Dimethyl sulfoxide (anhydrous) was supplied from Fluka. Minimum essential medium eagle (MEM), Dulbecco's modified eagle medium (DMEM) and trypsin were purchased from CellGro while fetal bovine serum (FBS) and penicillin-streptomycin from Gibco. All aqueous solutions are prepared in distilled, deionized water (Millipore, Synthesis System) with a resistivity of 18.2 Ωcm.

Preparation of nano-pH probe. Nanopipettes were fabricated from quartz capillaries with filament (QF100-70.7.5, Sutter Instrument). Prior to pulling, capillaries were treated with piranha solution (sulfuric acid:hydrogen peroxide, 3:1 v/v) (Caution: piranha solution′ reacts violently with organic materials and may become extremely hot when prepared.) and rinsed thoroughly with distilled water and 2-propanol. Treated capillaries were kept in 2-propanol until use to prevent contamination. Capillaries were pulled using a P-2000 laser puller (Sutter Instrument) with a two-line program with following parameters; Line 1: Heat 700, Fil 4, Vel 20, Del 170, Pull 0 and Line 2: Heat 680, Fil 4, Vel 40, Del 170, Pull 200. The resulting nanopipettes had a pore diameter of ˜97 nm detected by a FEI Quanta 3D field emission microscope. Nanopipettes were stored in a sealed box until modification. Nanopipettes were functionalized by backfilling 10 μl of 0.25% chitosan solution and centrifuged at 4000 rpm to assure the coverage of the nanopipette tip with chitosan matrix. After centrifugation, excess chitosan was aspirated and nanopipettes were left to air-dry overnight. Dried nanopipettes were backfilled with 10 mM phosphate buffer saline (PBS) solution at pH 7.0, then centrifuged to remove residual air bubbles trapped at the tip of nanopipettes. Once filled all nanopipettes were kept in 10 mM PBS (pH 7.0) until pH measurements to prevent clogging of the nanopore.

Sensing setup. To carry out analytical characterization experiments of chitosan-modified nanopipette sensors, a two-electrode setup connected to a potentiostat (1030C, CH Instruments Inc.) was used for sensing. A 125 μm platinum wire (Goodfellow Corporation) placed into nanopipettes filled with electrolyte served as the working electrode while a pseudo Ag/AgCl electrode placed in bulk solution (PBS or cell media) served as the reference electrode. Linear sweep voltammetry was utilized for all in vitro measurements with a scan rate of 0.1 V/sec.

Intracellular measurements were performed by combining the potentiostat and scanning ion conductance microscope (SICM) with a low-noise mechanical switch. The SICM setup consisted of an Axopatch 200B amplifier (Molecular Devices) for current feedback measurements, a MP-285 motorized micromanipulator (Sutter Instrument) for coarse positioning of the nano-pH probe, a piezo stage (NanoCube, Physik Instrumente) for fine positioning and insertion of the nano-pH probe sensors, and a programmable interface for hardware control of the setup. This system is run by custom software written in LabVIEW (National Instruments). All experiments with cells were conducted on an inverted fluorescence microscope (Olympus IX 70) equipped with an eyepiece camera (Dino-Eye, Big C).

Cell culture. HeLa, MCF-7, MDA-MB-231 and human fibroblast cells were cultured in a conditioned environment with 5% CO₂ and 90% humidity at 37° C. HeLa, MCF-7 and MDA-MB-231 cells were cultured in 1×MEM, while human fibroblasts in 1×DMEM. All media were supplemented with 10% FBS and 1% Penicillin-Streptomycin.

Fluorescence microscopy. MDA-MB-231 cell cultures were exposed to a pH-sensitive fluorescent indicator, BCECF-AM. The working solution was prepared to a concentration of 1 μM in Hank's Buffered Salt Solution (HBSS) and incubated at 37° C. for 15 min before fluorescent imaging. Cells were washed with Dulbecco's phosphate-buffered saline (DPBS) before loading of 1 μM BCECF-AM solution. After incubation, excess fluorescent dye was rinsed off the cells with HBSS was loaded on the culture for imaging.

For intracellular pH buffer calibration, cell cultures were exposed to complete pH calibration buffer prepared according to the protocol supplied with the Intracellular pH Calibration Buffer Kit (Life Technologies, P35379), and were incubated at 37° C. for 10 min before imaging. Intracellular pH calibration was done in three replicates. All fluorescence microscopy analyses were carried out with a Leica SP5 confocal microscope using the Leica Application Suite Advance Fluorescence (LAS AF 3) software. Further image analyses were performed with Fiji-ImageJ software.

### EXAMPLES

### Example 1: Characterization of pH-Responsive Quartz Nanopipette Sensors

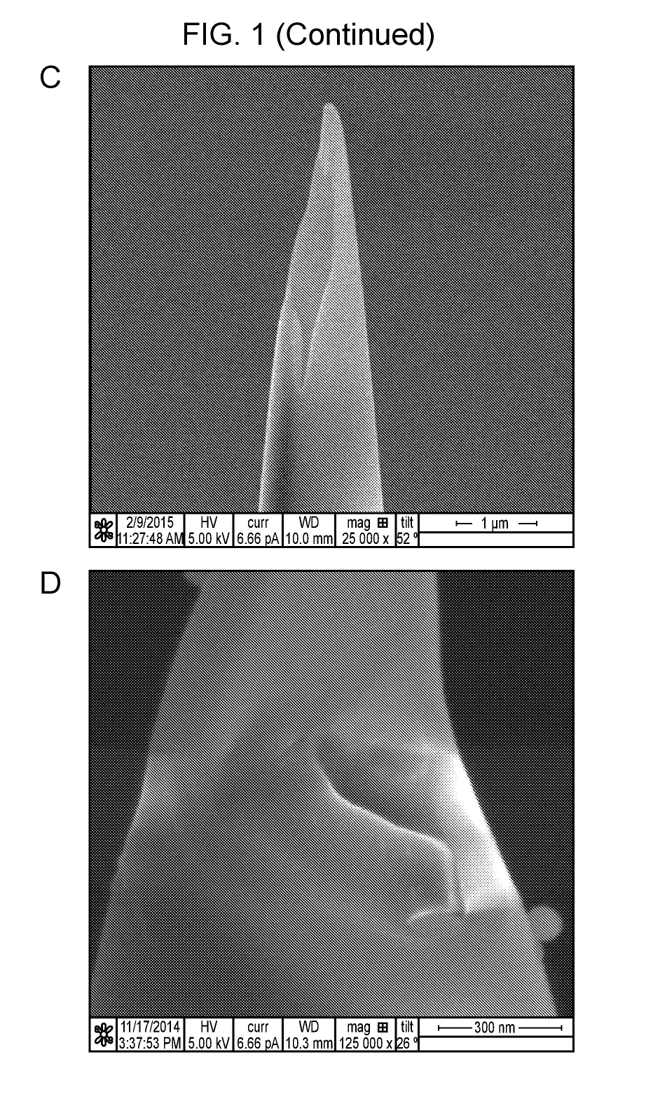

The measurement principle of nanopipettes is based on the ionic current at the tip. This ionic current is highly dependent on the pore size and surface charge of the nanopipette³⁴. The surface charge of a quartz nanopipette is negative due to dissociation of silanol groups at the glass-liquid interface. Quartz undergoes protonation at extremely acidic pH values⁴¹. These surface properties of quartz reduce pH sensing capabilities, making bare nanopipettes inappropriate for measuring very small pH changes. Limitations associated with the low sensitivity of bare quartz surfaces can be overcome through the incorporation of pH responsive polymeric entities onto nanopipette surfaces. Here, we employed chitosan as the pH sensitive surface coating. Chitosan, with a strong positive charge at acidic pH, is attracted to hydroxyl moieties on the negatively charged quartz surface through electrostatic interactions. In addition to alterations of the surface charge, the thickness of the chitosan layer has been shown to change with pH which may enhance the sensitivity of the nanopipette^{42,43}. To evaluate the presence and impact of the chitosan layer on the nanopipette surface, we monitored the changes in current responses as a result of surface modification. FIG. 1A demonstrates the electrochemical traces of the bare and chitosan-modified quartz nanopipettes filled with 10 mM PBS (pH 7.0) in the potential range of −0.5 to 0.5 V (vs. Ag/AgCl reference electrode). The recorded current response significantly decreases after chitosan modification. The typical geometric shape of a nanopipette tip is conical (FIG. 2A), and the pore size of quartz nanopipettes was determined by SEM and found to be ˜97 nm (FIG. 1B). Additional SEM micrographs were taken to further confirm the presence of the chitosan layer (FIG. 2B). Because the chitosan modification was done on the inside of the nanopipette, a focused ion beam was used to vertically etch the nanopipette and expose the internal surface. The cross-section image shows chitosan residues inside of the nanopipette surface when compared to that of a bare nanopipette (FIGS. 1C and D).

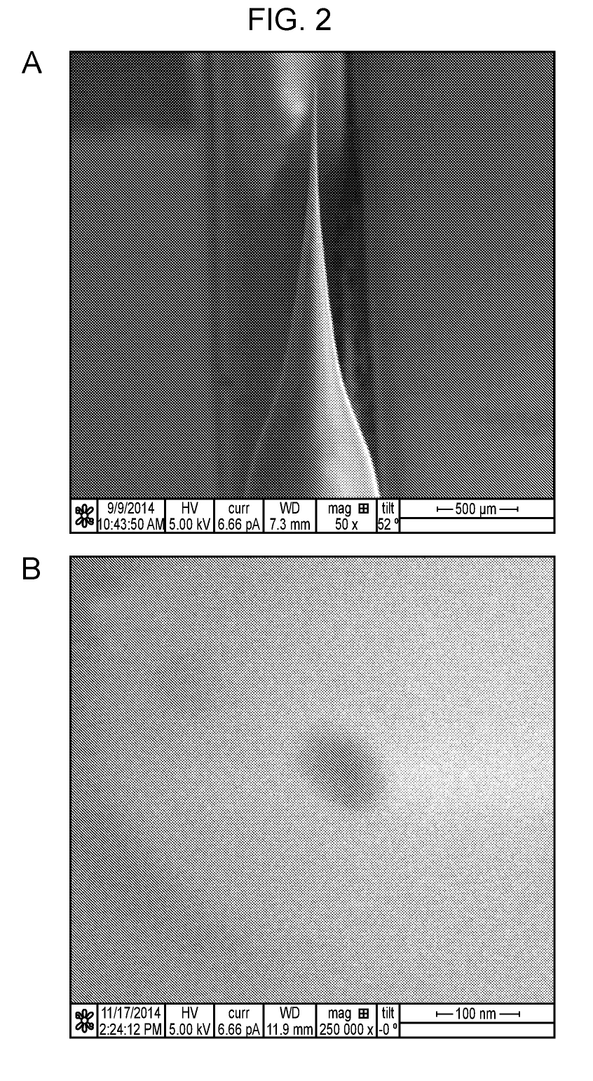

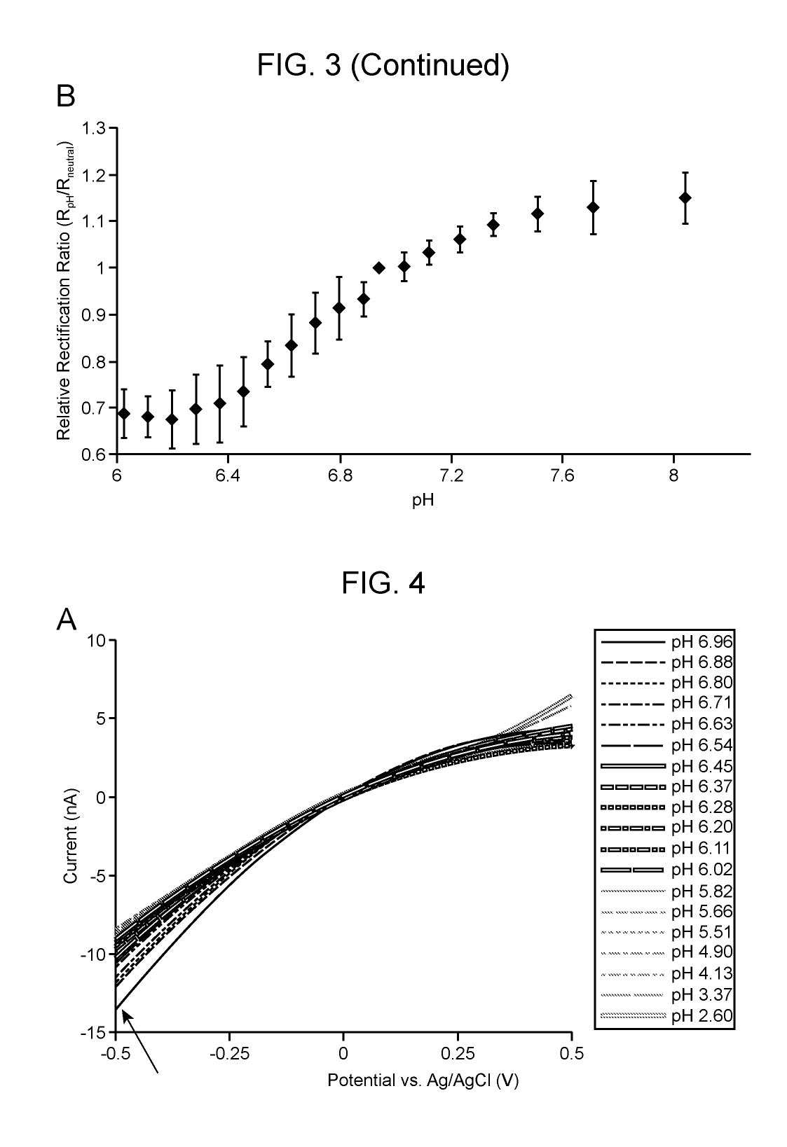

Once the presence of the chitosan layer was confirmed with SEM and electrochemistry, analytical characterization of the functionalized nanopipettes was conducted using linear sweep voltammetry. The potential range spanned from −0.5 to 0.5 V with a scan rate of 0.1 V/sec. The modulation of pH was achieved by a conventional acid-base titration approach. Calibration of chitosan-modified nanopipettes was performed by consecutive additions of 20 μl of first 1 M NaOH and second HCl into 0.1 M PBS (pH 7.0). Current rectification of modified nanopipettes at +/−0.5 V changed in response to the changing pH of the buffer solution, as expected with the alteration of charge on the chitosan layer. Chitosan contains a glucosamine residue on its polysaccharide backbone (pKₐ˜6.5) making chitosan pH-responsive³⁸. pH values below the pKₐ protonate the chitosan layer making the nanopipette surface positively charged, whereas basic conditions deprotonate chitosan's amine functional group, increasing the net negative charge at the surface (FIG. 3A). For quantitation of pH, a relative rectification ratio (RR) has been defined as R_{RR}=RR_{pH}/RRₙₑᵤₜᵣₐₗ where RR_{pH} and RRₙₑᵤₜᵣₐₗ are RR at a specific pH and at pH 7.0 respectively. FIG. 3B displays the calibration curve obtained by acid-base titrations using the chitosan-modified nanopipette within the physiologically relevant pH range from 6.02 to 8.04. The trend observed in the pH calibration curve is typical of isoelectric point determination experiments. A slight shift in the isoelectric point of chitosan may be due to the nanoscale conical geometry of the nanopipette tip, which can impede the uniform diffusion of ions. The sensitivity of the chitosan-functionalized pH-nanoprobe was 0.09 pH units. This high sensitivity to pH makes the nanoprobe a powerful tool for intracellular pH measurements. Current-potential curves of individual pH as well as a larger range pH calibration are given in FIG. 4A-4C. Bare nanopipettes were tested for pH sensing; as expected, these nanopipettes demonstrated low sensitivity towards pH changes (FIG. 5).

### Example 2: pH Sensing in Cell Culture Media

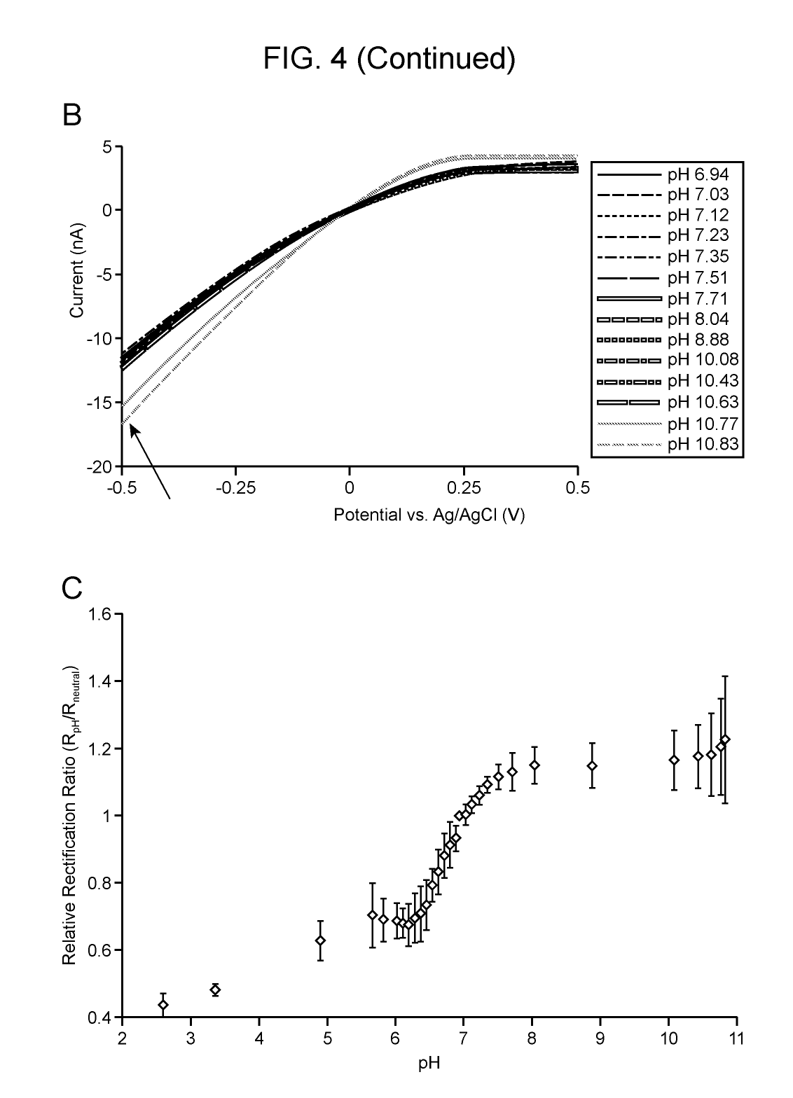

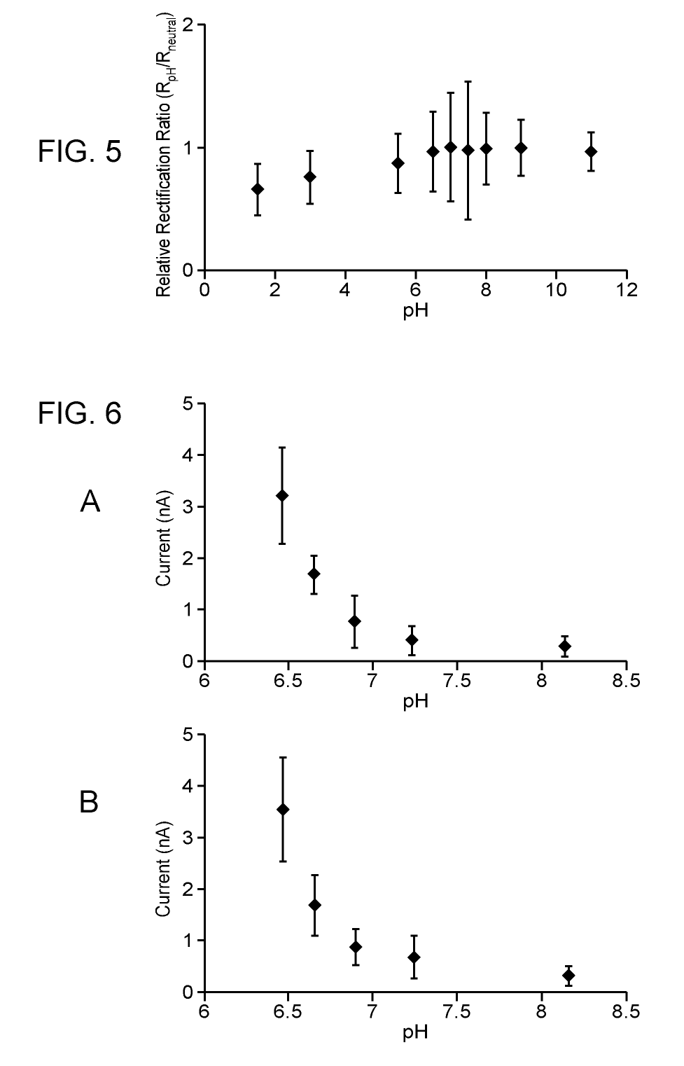

Our motivation for developing a solid nanopore pH probe is to measure intracellular pH at the single-cell level and to identify cancer cells with their distinctive metabolic characters. To perform intracellular pH measurements, chitosan-modified nanopipettes were further calibrated in cell culture media, MEM and DMEM. As cell media contain various amino acids, vitamins and other ingredients, optimum working parameters were different from those determined for PBS. The scanned potential range was from −0.2 to 0.6 V with a scan rate of 0.1 V/sec. The sensitivity of chitosan-modified nanopipettes for pH changes was the highest at 0.6 V. FIG. 6A-6B shows the calibration of nano-pH probes in 1×MEM and DMEM solution. Calibration of nano-pH probes in the media was carried out by consecutive additions of 20 μl of 0.1 M HCl. The measurements were done 15 sec after the addition of acid solution to cell culture media to obtain a homogeneous solution. Representative linear sweep voltammograms are demonstrated in FIG. 7A-7B for acid titration of MEM and DMEM media. As ingredients of these media are different, their buffering capacities are slightly different with DMEM being more resistant to pH changes compared to MEM.

### Example 3: Measurement of Intracellular pH of Cancerous and Non-Cancerous Cells

Direct measurement of intracellular pH is challenging due to the small size of cells and the complexity of the physiological matrix. While the physiological pH level is marginally alkaline, the intracellular pH level of individual cells in a large population and subcellular compartments is unknown. Conventionally, fluorescence dyes (e.g. BCECF-AM, oregon green) are utilized for indirect detection of pH in cells³⁰. Although these pH indicators reveal an approximation of pH over a large cell population, there are several disadvantages of using fluorescence dyes: i) low sensitivity due to short pH range, ii) fast photobleaching, iii) cytotoxicity. Additionally, accumulation of these dyes in certain organelles and their rate of leakage can result in incorrect interpretations. Our studies using a conventional pH indicator, BCECF-AM, to measure intracellular pH of MDA-MB-231 cells have proven the drawbacks of using fluorescence for accurate and sensitive evaluation of intracellular events. In these studies, in which fluorimetric intracellular pH measurements were made, cells were exposed to BCECF-AM and incubated for 15 min. Then, cells were washed and exposed to nigericine containing cellular pH calibration buffer (pH 7.5, 6.5, 5.5 and 4.5) for 10 min. BCECF-AM has dual excitation wavelengths; therefore, images were taken at 458 and 488 nm. Bright field and fluorescence micrographs were obtained for the two excitation wavelengths of each pH value. A ratiometric calibration curve was obtained using fluorescence intensities of 16 to 23 individual cells (data not shown). One group of cells served as negative control (without BCECF-AM) to evaluate the presence of intracellular autofluorescence. In the absence of the pH dye, there was no observable fluorescence for MDA-MB-231. Cells exposed to BCECF-AM were used to estimate the intracellular pH values of individual cells. The average intracellular pH value obtained from 10 individual cells was calculated to be 6.78 (±0.83). However, the micrographs taken after BCECF-AM exposure revealed that fluorescence intensity over the cell body varies (data not shown). Fluorescence intensity was higher where cells were thicker. Additionally, any two regions in close proximity to one another in an individual cell were found to have large variation in pH values. These variations can be attributed to (i) unequal distribution or accumulation of the fluorescence dye; (ii) cross-reactivity of the fluorescence dye with another molecule. Another drawback of fluorescence measurements is the sample preparation step that requires the frequent change of media, which can stress cells and alter the basal intracellular levels. Moreover, the use of fluorescence dyes does not allow continuous interrogation of a single cell over the course of treatment, such as in drug testing, or toxicity measurements, because the presence of these dyes along with the compound of interest may cause false experimental conclusions by changing the physiology of a cell or by cross-reacting with the compound to be tested. In other words, continuous interrogation of a single cell over the course of time for evaluating the cellular impact of therapeutics, channel activators, or toxins cannot be carried out with conventional fluorescence probes.

In order to directly and accurately measure intracellular pH, chitosan-modified nanopipettes were inserted in the cytoplasm of the cells in culture. We used this sensing technology, for the first time, for the direct monitoring of intracellular pH of human cancerous and non-cancerous cell lines, including human fibroblast, HeLa, MCF-7 and MDA-MB-231. Human fibroblast cells are selected as a non-cancerous model to investigate intracellular pH levels at normal cytoplasmic conditions. HeLa cell lines are the most commonly used human cancer type due to their rapid and continuous growth in cell culture. Additionally, because of reports of contamination and heterogeneity of HeLa cells, determination of the intracellular pH levels of these cells may allow us to evaluate the cell heterogeneity⁴⁴. MCF-7 and MDA-MB-231 are distinct breast cancer cell lines. MCF-7 is a hormone-responsive cell line and its growth is stimulated with estrogen; MDA-MB-231 derives from an invasive breast cancer which was found to be highly metastatic⁴⁵. We chose to interrogate these two breast cancer cell lines because they exhibit different drug sensitivities and we sought to determine whether this could be correlated with differences in the intracellular pH levels.

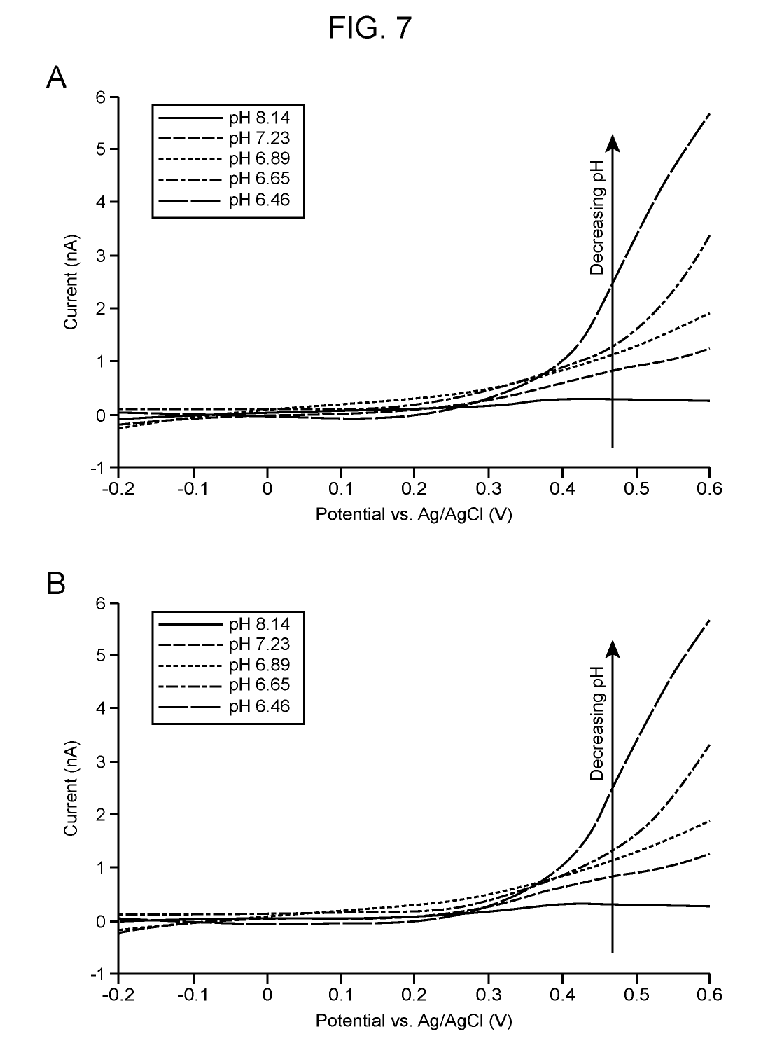

Chitosan-modified nanopipettes were inserted to individual cells using a customized scanning ion conductance microscope which detects current feedback for positioning the nanopipettes. Recently we have demonstrated that this custom-built platform can perform nanobiopsies at the single-cell level for genomic investigations⁴⁶. FIG. 8A demonstrates a representative feedback signal recorded during the approach-penetration-retraction process of chitosan-modified nanopipettes. After the insertion of the nano-pH probe into a cell, linear sweep voltammograms were recorded and the current-responses at a bias potential of 0.6 V were used to calculate the intracellular pH levels of single cells.

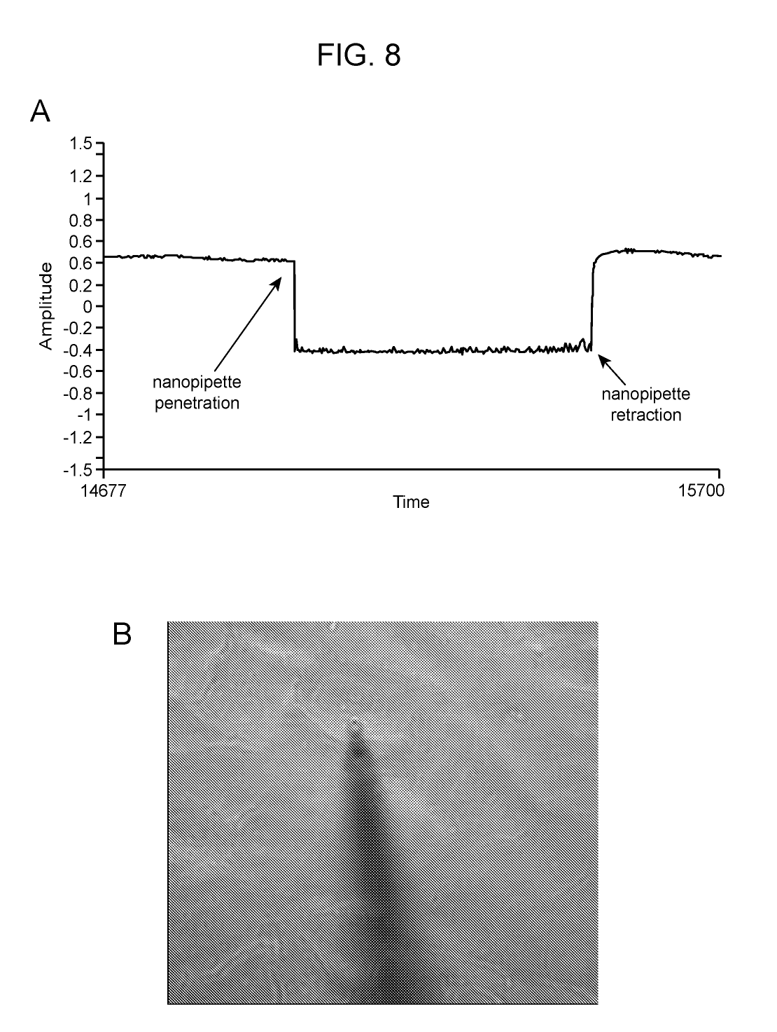

From voltammetric current responses at 0.6 V versus Ag/AgCl, the calculated intracellular pH levels of individual cells and average pH values for all cell lines are shown in FIG. 9A-9D. Seven human fibroblast cells were interrogated for intracellular pH and the average pH was 7.37±0.29 (FIG. 9A). The observed intracellular pH level in these human fibroblasts is in line with previous reports estimating pH levels through indirect and destructive approaches, including monitoring of ion exchangers (NHEs and NBCs) and acid transporters (AEs)¹⁷.

We also used the nano-pH probes to investigate the metabolic differences between non-cancerous and cancerous cells. As cancer cells have a faster metabolic rate compared to non-cancerous cells, production of acidic species and CO₂ in cancer cells is higher as well. Using the chitosan-modified nano-pH probe in 14 individual HeLa cells for intracellular pH measurements, we found the average pH for HeLa cells to be 6.75±0.27 (FIG. 9B).

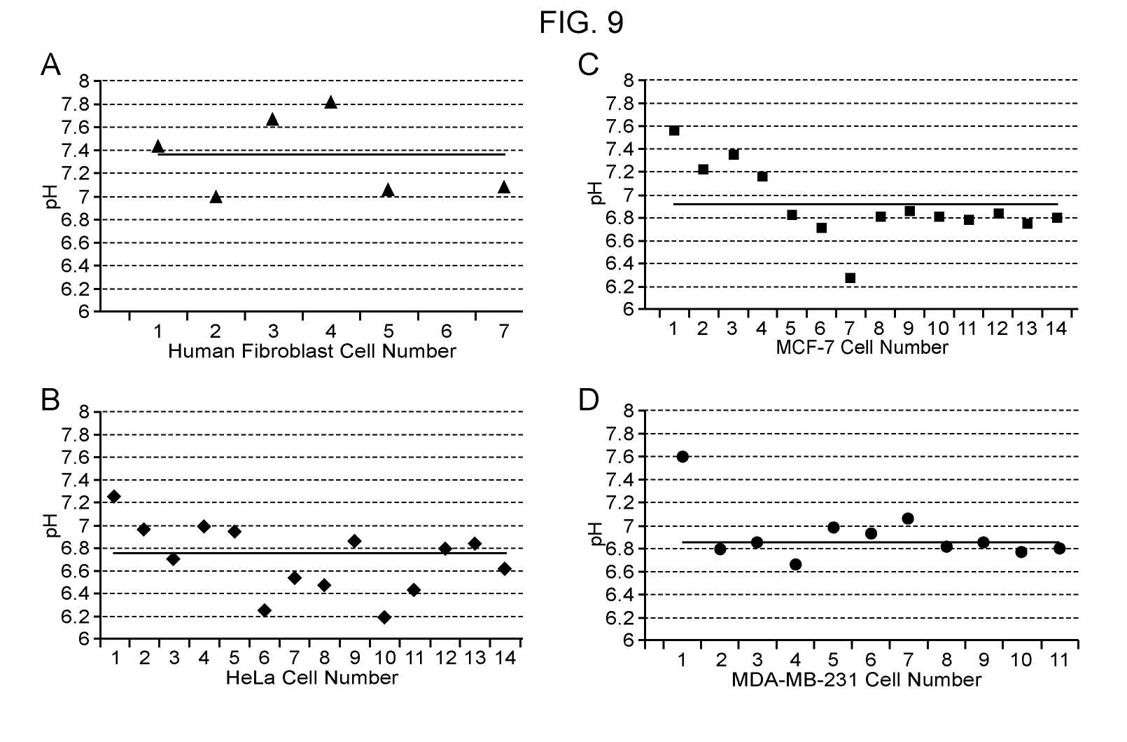

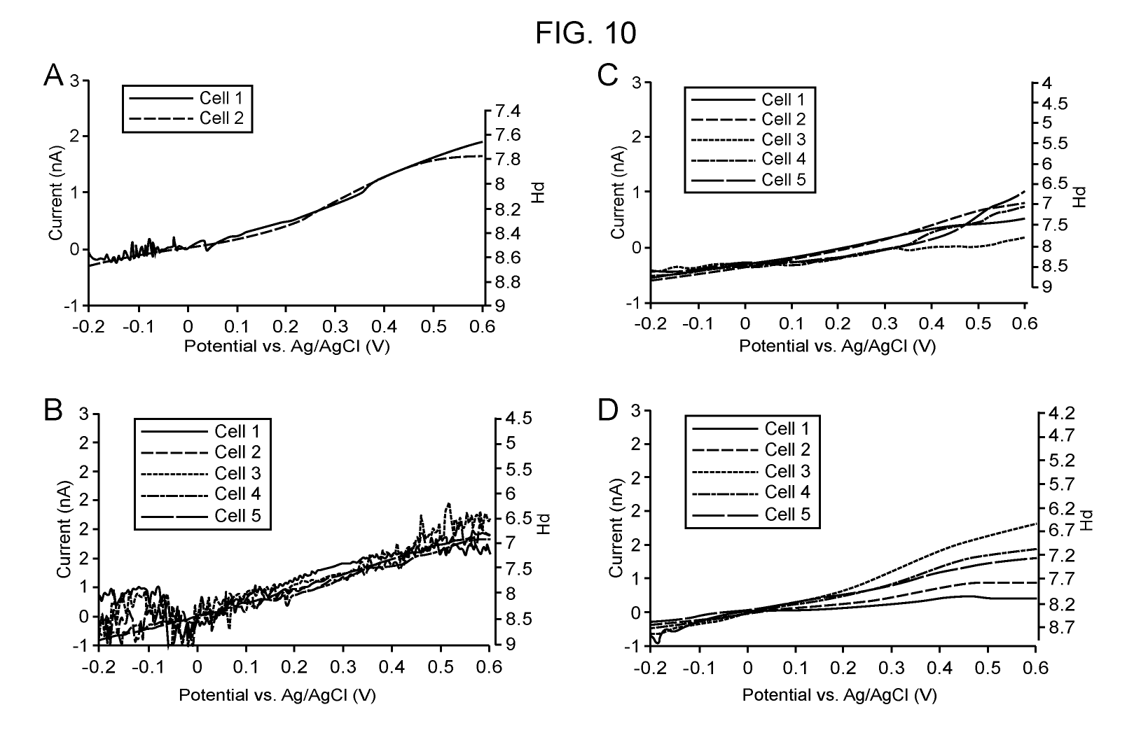

To compare whether a similarly acidic intracellular environment is present in other cancer cell lines, we performed pH measurements on breast cancer lines. Using the nano-pH probe, was observed an average intracellular pH level for 14 individual MCF-7 cells of 6.91±0.20 (FIG. 9C). The average intracellular pH was found to be 6.85±0.11 for MDA-MB-231 using 11 individual cells (FIG. 9D). Representative linear sweep voltammograms of individual cell measurements are given in FIG. 10A-10D. Our data demonstrate that the intracellular environment can differ from cell to cell in a way that is detectible by pH. These differences can be attributed to different metabolic speeds of individual cells and may be used for the identification of heterogeneous cells in a large population, such as tumors. The small tip size of the nano-pH probe reduces the damage during insertion (FIG. 11A-11C, compare micrographs in 11A and 11B) and measurement. This aspect enables continuous or intermittent interrogation of the same cell over the course of pharmaceutical manipulations and drug therapies (see next section). FIG. 11C illustrates regeneration and reusability of nano-pH probes for consecutive in vitro measurements. pH probes were tested after cell interrogations in 0.1 M PBS (pH 7.0). Additionally, this test is important to control the integrity of the probe after use for in vitro measurement.

To more fully deploy the pH nano-probe described herein, one builds a fully-automated high-throughput robotic system that will allow us to interrogate hundreds of cells in a range of minutes. Cells having lower or higher pH values compared to the general population of cells will be identified and then tagged with a molecular marker to nanobiopsy for DNA and RNA sequencing.

### Example 4: Pharmaceutical Manipulation of Intracellular pH

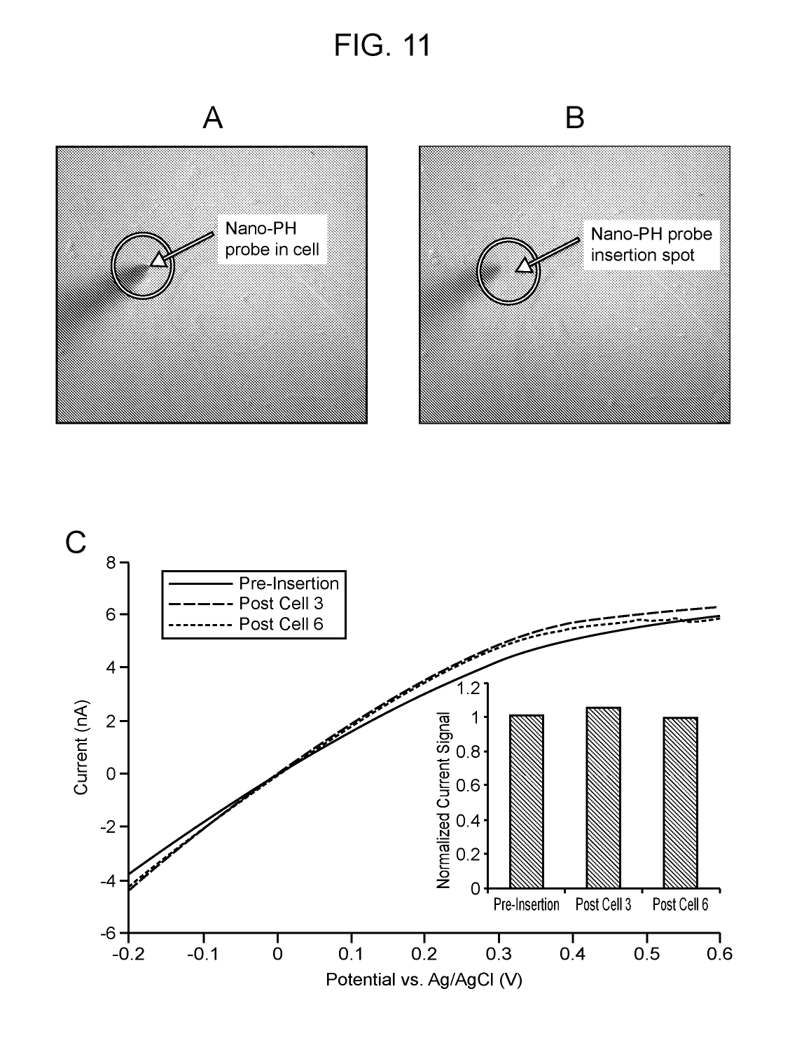

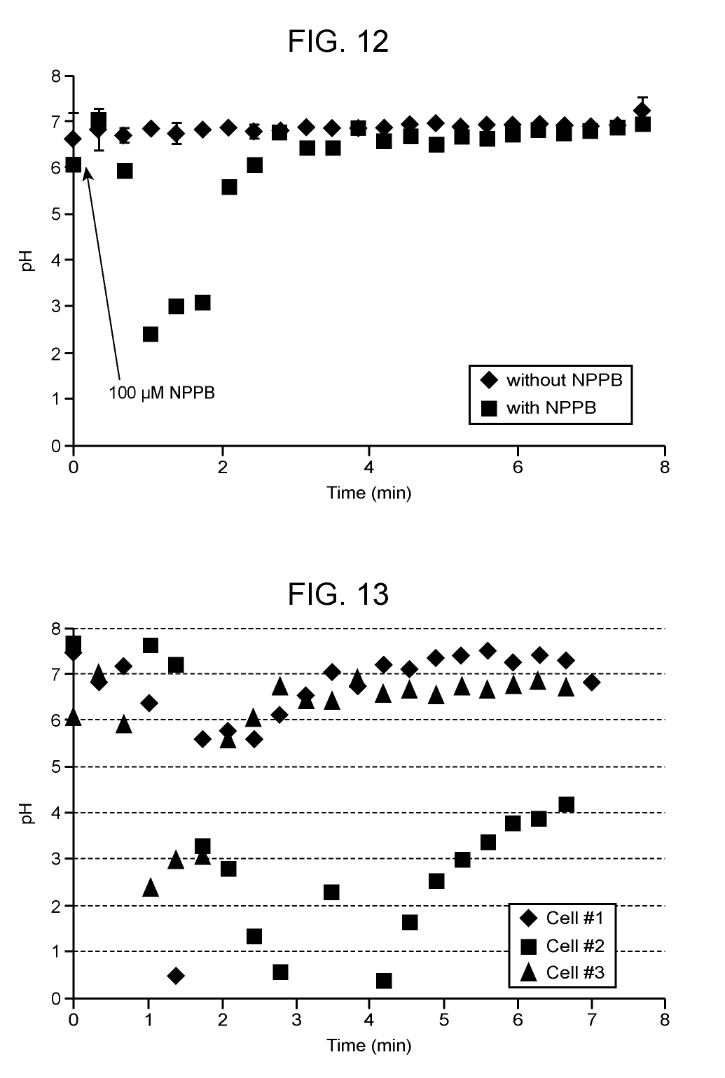

The present nano-pH probe can be used to monitor intracellular pH changes during drug therapy. To this end, the present nano-pH probe was arranged for continuous monitoring at a single cell during the addition of a known chloride channel blocker, 5-nitro-2-(3-phenylpropylamino)-benzoate (NPPB). NPPB has been shown previously to block chloride channels in renal epithelial and macrophage cells, with a resulting increase in acidity of the intracellular environment. Conventionally, the change in pH has been measured indirectly by introduction of fluorescent dye (BCECF-AM)^{47,48}. Thus, this pharmaceutical manipulation test not only serves to demonstrate the capability of real-time measurement of nano-pH probes but also the specificity towards pH detection. To obtain a baseline, nano-pH probes were inserted in MDA-MB-231 cells and consecutive pH measurements were performed for every 21 second for 7 min. This real-time pH monitoring in MDA-MB-231 cells showed minimal drift around a value 7 over the course of measurement (FIG. 12, diamonds). To study the effect of NPPB, a nano-pH probe was inserted into MDA-MB-231 cells and intracellular pH recording was initiated just prior to the addition of 100 μM of NPPB (freshly prepared in anhydrous DMSO) to the cell media. The squares in FIG. 12 display the pH changes as a result of NPPB exposure over a 7 min time period. The intracellular pH level dropped significantly within the first 2 min after the introduction of NPPB and went as low as 2.5. The measured pH levels stabilized by 4 min post NPPB introduction. This increase in pH can be due to apoptosis resulting in the shrinkage of cell body, which would expose the tip of the nano-pH probe to the cell media. Intracellular pH measurements of three individual MDA-MB-231 cells with the nano-pH probe not only showed the real-time pH changes after NPPB exposure but also the variations from cell to cell in terms of drug-response (FIG. 13).

### Example 5: Detection of Redox Changes in a Cell

The device described above can be further modified with a layer attached to the chitosan layer on the nanopipette that is responsive to oxidation or reduction of components in the cell.

The above-described chitosan-modified quartz nanopipettes can be modified with immobilized proteins such as hemeproteins and enzymes. This immobilization to chitosan can be realized through either a peptide bond formation mechanism or catalytic reactions as chitosan possesses carboxylic groups and randomly distributed glucosamine residues on its polymeric backbone. Immobilization of redox active small proteins onto the chitosan layer makes the so-functionalized nanopipette sensitive to highly reactive radicals such as reactive oxygen (ROS) and nitrogen species (RNS), and hydrogen peroxide. For details on ROS, see Salehi, et al., “Hemeproteins including hemoglobin, myoglobin, neuroglobin, cytoglobin and leghemoglobin,” J. Photochemistry and Photobiology B: Biology 133 11-178 (2014).

These radical (e.g. reactive oxygen species) are known to contribute to many disease states, such as cancer, aging, stroke, Parkinson's and Alzheimer's diseases. Therefore the measurement of physiological levels of ROS and RNS is of great importance.

In the presence of ROS or RNS, redox sensitive surface functionalities inside the nanopipette undergo either reduction or oxidation depending upon the oxidation states. Such changes in the oxidation state result in a change of the surface charge. The change in surface charge is in correlation with the amount of ROS or RNS present in the aqueous environment. The detection of reactive species is done by measuring the fluctuations of ionic current at the nanopore when a potential difference is applied across the quartz nanopore.

### Example 6: Multiplex Array of Nano-pH Probes

The device described above can be further constructed in a multiplexed array of nano-pH probes. In addition, a number of surface recognition materials can be added to the interior of various nanopipettes used in the array. The number nanopipette structures can be varied, and not all of them may contain the chitosan pH sensing coating.

One possible method for making conical nanopipette structures for use in this array is described in Meyyappan, U.S. Pat. No. 9,182,394. This patent describes an array of nanopipette channels, formed and controlled in a metal-like material that supports anodization. As described there, a thin substrate of anodizable metal such as Al, Mg, Zn, Ti, Ta and/or Nb is anodized at temperature T=20-200° C. in a chemical bath of pH=4-6 and electrical potential 1-300 Volts, to produce an array of anodized nanopipette channels, having diameters 10-50 nm, with oxidized channel surfaces of thickness 5-20 nm. A portion of exposed non-oxidized anodizable metal between adjacent nanopipette channels, of length 1-5 μm, is etched away, exposing inner and outer surfaces of a nanopipette channel.

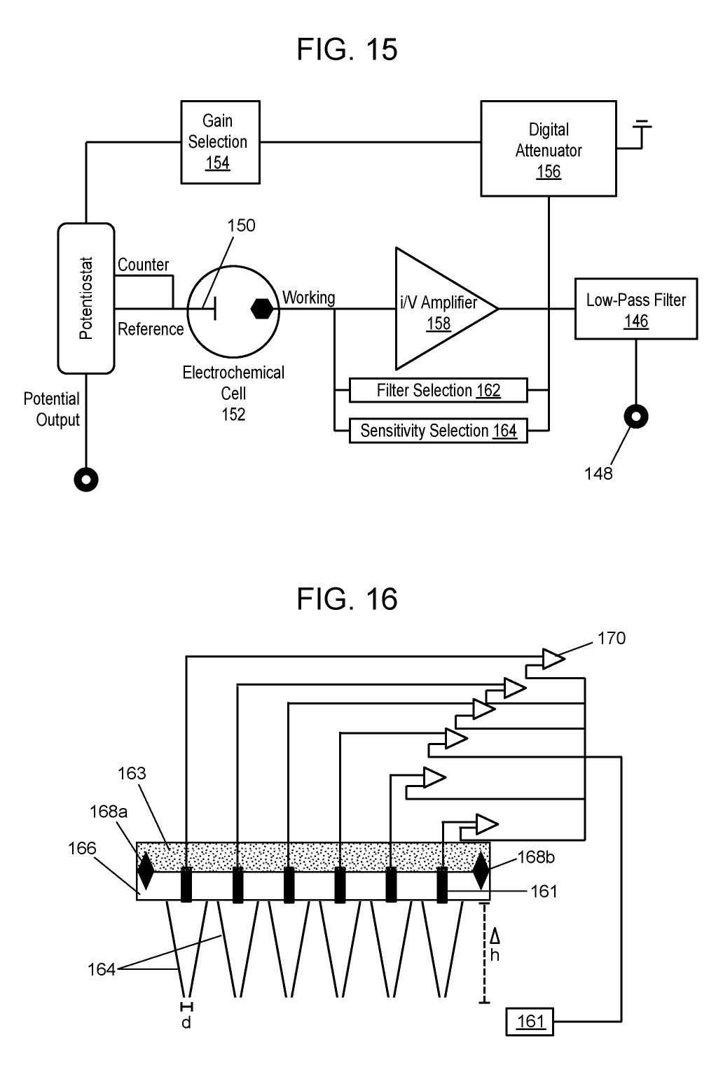

FIG. 16 schematically displays a two dimensional sectional view of a nanoprobe array. FIG. 16 shows six nanopipette probes, for purposes of illustration. A much larger array can be used. An individual nano-pH probe comprises a nanopipette containing a conductive material and connected to a working (sensing) electrode 161 which extends into the interior of the nanopipette. An insulating layer 166 is applied to the back portion of the array of nanopipettes 164, constructed, as described above, e.g., as crystalline SiO₂. An inactive support structure 163 is attached to the insulating layer 166 and serves to support the insulation and the electrode array. Each nanopipette in the array 164 extends a distance from the insulating layer to a height of Ah, as shown, and has a tip opening of diameter d. The diameter of nanopores (d) can be between 5 and 200 nm, and the length of nanopipette dimension Ah can be between 10 and 400 μm. Each working electrode 161, is connected to an input of an individual amplifier 170, which has a differential input from an individual probe in the array 164, which contains conductive material within a nanopipette. An individual signal amplifier 170 is provided for each nanopipette, and outputs (connection not shown) to a measuring device with a readout of sensitive pH changes in a cell, such as shown in FIG. 15. The nanopipettes in the array 164 are fabricated on a perforated insulating layer 166 made, e.g., of oxidized aluminium. The perforations are for insertion of sensing electrodes with a size range of 5 to 125 μm.

In addition, magnetic structures 168a, 168b are provided to provide a removable attachment between the support structure 163 and the insulating layer 166. This provides access to nanopipettes in the array and allows modification of pipettes, as well as filling them with the supporting electrolyte.

The modification is done prior to insertion of the electrodes (161) by casting the inner surface of pipette structures with polymers or recognition molecules. The surface coating process can be performed for the entire inner surface but not necessarily since the ionic current changes are dominated by the first 0.1 to 5 μm of the nanopore.

These surface recognition materials can be polymers including Nafion®, phenylenediamine, poly-1-lysine, poly-acrylic acid and polypyrrole; enzymes including oxidoreductase and dehydrogenase families; proteins including avidin and prion; and antigens, RNA fragments and aptamers. These substances can be utilized alone or in combination for the functionalization of individual or an array of nanoprobes for targeted sensing purposes. The surface modification protocols must be optimized for each recognition material including surface chemistry for immobilization, concentration, incubation time and temperature. The nanopipette filling solution's properties such as pH, electrolyte type and concentration for each sensing array should be evaluated for the highest detection sensitivity.

After necessary surface modifications are completed and filling electrolyte is introduced, a customized printed circuit board (PCB) with built-in sensing electrodes is placed on top of the nanopipette array by aligning the electrodes to perforations. Sensing electrodes are metallic including silver, platinum, gold; or redox-based (Silver-silver(I)chloride) or non-metals including glassy carbon, graphite and boron-doped diamond. When electronics are inserted into the nanopipette array, inner components of the nanopipette array are completely sealed. Magnetic structures 168 made of neodymium ensure both electronics and the nanopipette array are locked to each other. The electronic structure of this invention contains all the necessary circuitry for individual channels and is able to perform synchronized or customized sensing.

### CONCLUSION

The above specific description is meant to exemplify and illustrate the invention and should not be seen as limiting the scope of the invention, which is defined by the literal and equivalent scope of the appended claims. Any patents or publications mentioned in this specification are intended to convey details of methods and materials useful in carrying out certain aspects of the invention which may not be explicitly set out but which would be understood by workers in the field. Such patents or publications are hereby incorporated by reference to the same extent as if each was specifically and individually incorporated by reference and contained herein, as needed for the purpose of describing and enabling the method or material referred to.

### REFERENCES

1. Higgins, C. F. Multiple molecular mechanisms for multidrug resistance transporters. Nature 446, 749-757 (2007).
2. Videira, M., Reis, R. L. & Brito, M. A. Deconstructing breast cancer cell biology and the mechanisms of multidrug resistance. Biochimica et Biophysica Acta (BBA)—Reviews on Cancer 1846, 312-325, doi:http (colon slash slash) dx.doi.org/10.1016/j.bbcan.2014.07.011 (2014).
3. Lee, M.-C. W. et al. Single-cell analyses of transcriptional heterogeneity during drug tolerance transition in cancer cells by RNA sequencing. Proceedings of the National Academy of Sciences 111, E4726-E4735, doi:10.1073/pnas.1404656111 (2014).
4. Workman, P. Genomics and the second golden era of cancer drug development. Molecular BioSystems 1, 17-26, doi:10.1039/b501751n (2005).
5. Garraway, L. A. & Jänne, P. A. Circumventing Cancer Drug Resistance in the Era of Personalized Medicine. Cancer Discovery 2, 214-226, doi:10.1158/2159-8290.cd-12-0012 (2012).
6. Yap, T. A., Gerlinger, M., Futreal, P. A., Pusztai, L. & Swanton, C. Intratumor Heterogeneity: Seeing the Wood for the Trees. Science Translational Medicine 4, 127ps110, doi:10.1126/scitranslmed.3003854 (2012).
7. Gerlinger, M. et al. Intratumor Heterogeneity and Branched Evolution Revealed by Multiregion Sequencing. New England Journal of Medicine 366, 883-892, doi:doi:10.1056/NEJMoa1113205 (2012).
8. Zhang, J. et al. Intratumor heterogeneity in localized lung adenocarcinomas delineated by multiregion sequencing. Science 346, 256-259, doi:10.1126/science.1256930 (2014).
9. Beltran, H. et al. Targeted Next-generation Sequencing of Advanced Prostate Cancer Identifies Potential Therapeutic Targets and Disease Heterogeneity. European Urology 63, 920-926, doi:http (colon slash slash) dx.doi.org/10.1016/j.eururo.2012.08.053 (2013).
10. Wu, T., Sempos, C. T., Freudenheim, J. L., Muti, P. & Smit, E. Serum iron, copper and zinc concentrations and risk of cancer mortality in US adults. Annals of Epidemiology 14, 195-201, doi:http (colon slash slash) dx.doi.org/10.1016/S1047-2797(03)00119-4 (2004).
11. Wiseman, H. & Halliwell, B. Damage to DNA by reactive oxygen and nitrogen species: role in inflammatory disease and progression to cancer. Biochem. J. 313, 17-29 (1996).
12. Zheng, G., Patolsky, F., Cui, Y., Wang, W. U. & Lieber, C. M. Multiplexed electrical detection of cancer markers with nanowire sensor arrays. Nat Biotech 23, 1294-1301, doi:http (colon slash slash) www.nature.com/nbt/journal/v23/n10/suppinfo/nbt1138_S1.html (2005).
13. Raghunand, N. & Gillies, R. J. pH and drug resistance in tumors. Drug Resistance Updates 3, 39-47, doi:http (colon slash slash) dx.doi.org/10.1054/drup.2000.0119 (2000).
14. Paroutis, P., Touret, N. & Grinstein, S. The pH of the Secretory Pathway: Measurement, Determinants, and Regulation. Vol. 19 (2004).
15. Weisz, O. A. Organelle Acidification and Disease. Traffic 4, 57-64, doi:10.1034/j.1600-0854.2003.40201.x (2003).
16. Gottlieb, R. A., Nordberg, J., Skowronski, E. & Babior, B. M. Apoptosis induced in Jurkat cells by several agents is preceded by intracellular acidification. Proceedings of the National Academy of Sciences 93, 654-658 (1996).
17. Casey, J. R., Grinstein, S. & Orlowski, J. Sensors and regulators of intracellular pH. Nat Rev Mol Cell Biol 11, 50-61, doi:http (colon slash slash) www.nature.com/nrm/journal/v11/nl/suppinfo/nrm2820 S1.html (2010).
18. Mahnensmith, R. L. & Aronson, P. S. The plasma membrane sodium-hydrogen exchanger and its role in physiological and pathophysiological processes. Circulation Research 56, 773-788, doi:10.1161/01.res.56.6.773 (1985).
19. Damaghi, M., Wojtkowiak, J. W. & Gillies, R. J. pH Sensing and Regulation in Cancer. Frontiers in Physiology 4, doi:10.3389/fphys.2013.00370 (2013).
20. Stock, C. & Schwab, A. Protons make tumor cells move like clockwork. Pflugers Arch—Eur J Physiol 458, 981-992, doi:10.1007/s00424-009-0677-8 (2009).
21. Parks, S. K., Chiche, J. & Pouyssegur, J. pH control mechanisms of tumor survival and growth. Journal of Cellular Physiology 226, 299-308, doi:10.1002/jcp.22400 (2011).
22. Gatenby, R. A. & Gillies, R. J. Why do cancers have high aerobic glycolysis? Nat Rev Cancer 4, 891-899 (2004).
23. Rango, M., Bonifati, C. & Bresolin, N. Parkinson's disease and brain mitochondrial dysfunction: a functional phosphorus magnetic resonance spectroscopy study. J Cereb Blood Flow Metab 26, 283-290 (2005).
24. Timofeev, I., Nortje, J., Al-Rawi, P. G., Hutchinson, P. J. A. & Gupta, A. K. Extracellular brain pH with or without hypoxia is a marker of profound metabolic derangement and increased mortality after traumatic brain injury. J Cereb Blood Flow Metab 33, 422-427 (2013).
25. Hashim, A. I., Zhang, X., Wojtkowiak, J. W., Martinez, G. V. & Gillies, R. J. Imaging pH and metastasis. NMR in Biomedicine 24, 582-591, doi:10.1002/nbm.1644 (2011).
26. Carter, N. W. Intracellular pH. Kidney Int 1, 341-346 (1972).
27. Mabe, H., Blomqvist, P. & Siesjo, B. K. Intracellular pH in the Brain Following Transient Ischemia. J Cereb Blood Flow Metab 3, 109-114 (1983).
28. Lucien, F., Harper, K., Pelletier, P.-P., Volkov, L. & Dubois, C. M. Simultaneous pH Measurement in Endocytic and Cytosolic Compartments in Living Cells using Confocal Microscopy. e51395, doi:doi:10.3791/51395 (2014).
29. Talley, C. E., Jusinski, L., Hollars, C. W., Lane, S. M. & Huser, T. Intracellular pH Sensors Based on Surface-Enhanced Raman Scattering. Analytical Chemistry 76, 7064-7068, doi:10.1021/ac049093j (2004).
30. McNamara, K. P. et al. Synthesis, Characterization, and Application of Fluorescence Sensing Lipobeads for Intracellular pH Measurements. Analytical Chemistry 73, 3240-3246, doi:10.1021/ac0102314 (2001).
31. Peng, H.-s., Stolwijk, J. A., Sun, L.-N., Wegener, J. & Wolfbeis, O. S. A Nanogel for Ratiometric Fluorescent Sensing of Intracellular pH Values. Angewandte Chemie 122, 4342-4345, doi:10.1002/ange.200906926 (2010).
32. Actis, P. et al. Voltage-Controlled Metal Binding on Polyelectrolyte-Functionalized Nanopores. Langmuir 27, 6528-6533, doi:10.1021/1a2005612 (2011).
33. Actis, P., Mak, A. & Pourmand, N. Functionalized nanopipettes: toward label-free, single cell biosensors. Bioanal Rev 1, 177-185, doi:10.1007/s12566-010-0013-y (2010).
34. Umehara, S., Karhanek, M., Davis, R. W. & Pourmand, N. Label-free biosensing with functionalized nanopipette probes. Proceedings of the National Academy of Sciences 106, 4611-4616, doi:10.1073/pnas.0900306106 (2009).
35. Vilozny, B., Actis, P., Seger, R. A., Vallmajo-Martin, Q. & Pourmand, N. Reversible Cation Response with a Protein-Modified Nanopipette. Analytical Chemistry 83, 6121-6126, doi:10.1021/ac201322v (2011).
36. Karhanek, M., Kemp, J. T., Pourmand, N., Davis, R. W. & Webb, C. D. Single DNA Molecule Detection Using Nanopipettes and Nanoparticles. Nano Letters 5, 403-407, doi:10.1021/n10480464 (2005).
37. Vilozny, B. et al. Carbohydrate-actuated nanofluidic diode: switchable current rectification in a nanopipette. Nanoscale 5, 9214-9221 (2013).
38. Ozel, R. E., Wallace, K. N. & Andreescu, S. Chitosan coated carbon fiber microelectrode for selective in vivo detection of neurotransmitters in live zebrafish embryos. Analytica chimica acta 695, 89-95, doi:10.1016/j.aca.2011.03.057 (2011).
39. Ozel, R. E., Hayat, A., Wallace, K. N. & Andreescu, S. Effect of cerium oxide nanoparticles on intestinal serotonin in zebrafish. RSC advances 3, 15298-15309, doi:10.1039/C3RA41739E (2013).
40. Ozel, R. E., Ispas, C., Ganesana, M., Leiter, J. C. & Andreescu, S. Glutamate oxidase biosensor based on mixed ceria and titania nanoparticles for the detection of glutamate in hypoxic environments. Biosensors & bioelectronics 52, 397-402, doi:10.1016/j.bios.2013.08.054 (2014).
41. Behrens, S. H. & Grier, D. G. The charge of glass and silica surfaces. The Journal of Chemical Physics 115, 6716-6721, doi:doi:http (colon slash slash) dx.doi.org/10.1063/1.1404988 (2001).
42. Claesson, P. M. & Ninham, B. W. pH-dependent interactions between adsorbed chitosan layers. Langmuir 8, 1406-1412, doi:10.1021/100041a027 (1992).
43. Lee, H.-S., Eckmann, D. M., Lee, D., Hickok, N. J. & Composto, R. J. Symmetric pH-Dependent Swelling and Antibacterial Properties of Chitosan Brushes. Langmuir 27, 12458-12465, doi:10.1021/1a202616u (2011).
44. Masters, J. R. HeLa cells 50 years on: the good, the bad and the ugly. Nat Rev Cancer 2, 315-319 (2002).
45. Holliday, D. & Speirs, V. Choosing the right cell line for breast cancer research. Breast Cancer Research 13, 215 (2011).
46. Actis, P. et al. Compartmental Genomics in Living Cells Revealed by Single-Cell Nanobiopsy. ACS Nano 8, 546-553, doi:10.1021/nn405097u (2013).
47. Brown, C. D. A. & Dudley, A. J. Chloride channel blockers decrease intracellular pH in cultured renal epithelial LLC-PK1 cells. British Journal of Pharmacology 118, 443-444, doi:10.1111/j.1476-5381.1996.tb15422.x (1996).
48. Lukacs, G. L., Nanda, A., Rotstein, O. D. & Grinstein, S. The chloride channel blocker 5-nitro-2-(3-phenylpropyl-amino) benzoic acid (NPPB) uncouples mitochondria and increases the proton permeability of the plasma membrane in phagocytic cells. FEBS Letters 288, 17-20, doi:http (colon slash slash) dx.doi.org/10.1016/0014-5793(91)80992-C(1991).

What is claimed is:

1. A device for measuring pH inside a single cell, comprising:
(a) a nanopipette structure that (i) is operatively connectable to a micromanipulator and sensing device for piercing a cell on a support, (ii) contains a working electrode therein, said (iii) contains a polymer coating that selectively absorbs hydrogen ions;
(b) said nanopipette structure further connected to an amplifier circuit constructed to apply different voltages between the working electrode and a reference electrode in a solution and further constructed to measure an ionic current between the working electrode and the reference electrode under different voltages; and
(c) logic means for correlating different ionic currents measured by said amplifier circuit with pH values within a cell outside the nanopipette structure.

2. A device of claim 1 wherein said micromanipulator and sensing device comprises an SICM (scanning ion conductance microscope) and xyz controller controlling the nanopipette for movement to and into a single cell.

3. A device of claim 1 or 2 wherein said amplifying circuit comprises a detection circuit with gain controls and with a low pass filter for detecting ionic currents.

4. A device of claim 1 or 2 comprising an array of nanopipette structures connected to a single logic means.

5. A device of claim 4 wherein the chitosan has a monomer number between about 30,000 and 60,000 units.

6. The device of claim 5 wherein the chitosan comprises a hemeprotein attached thereto.

7. A device of claim 1 wherein the polymer coating is selected from the group consisting of sulfonated tetrafluorethylene copolymer (Nafion®), poly-1-lysine, and alginate.

8. The device of claim 1 wherein the amplifier circuit comprises a potentiostat connected to the reference electrode and responsive to input from an amplifier having an input from the working electrode.

9. The device of claim 8 wherein the potentiostat is connected to a counter electrode that is also connected to the potentiostat's reference electrode.

10. The device of claim 8 wherein the working electrode and the counter electrode are Ag/AgCl.

11. A device for measuring pH inside a single cell, comprising:
(a) a nanopipette electrically connected to a circuit that measures ionic current versus potential at various potentials and is attached to an insertion device for inserting the nanopipette into a single cell;
(b) logic means for correlating a rectification value with known pH values, wherein a rectification value obtained in a cell can be correlated with a known rectification value, thereby providing an output identifying a measured pH value;
(c) said nanopipette having a layer of chitosan material directly bound to the surface of the nanopipette and porous to hydrogen ions; and
(d) a circuit comprising a reference electrode that also functions as an auxiliary electrode and is connected to a potentiostat.

12. A device of claim 11 wherein the logic means is programmed for scanning the potential of the working electrode at a given potential range with respect to the reference electrode by measuring the current at an auxiliary electrode.

13. A device of claim 11 comprising an i/V amplifier that is bridged by a filter selection and a sensitivity selection circuit, wherein the components are adjusted to adjust the detectable current range based on the current passing through the electrolyte solution.

14. A method for making a device for measuring pH inside a single cell, comprising:
(a) preparing a nanopipette structure that (i) is operatively connectable to a micromanipulator and sensing device for piercing a cell on a support, (ii) contains a working electrode therein, and (iii) contains a polymer coating that selectively absorbs hydrogen ions;
(b) connecting said nanopipette structure to an amplifier circuit constructed to apply different voltages between the working electrode and a reference electrode in a solution and further constructed to measure an ionic current between the working electrode and the reference electrode under different voltages; and
(c) connecting said nanopipette structure to logic means for correlating different ionic currents measured by said amplifier circuit with pH values within a cell outside the nanopipette structure.

15. The method of claim 14 wherein said polymer coating is applied by binding a chitosan material layer to the nanopipette; further comprising connecting said working electrode to an amplifier that conducts and measures an I-V curve for ionic current through the nanopipette.

16. A method of measuring pH in a cell, comprising:
(a) providing a nanopipette structure, having an interior layer responsive to pH ions, and being electrically connected by a working electrode to a circuit comprising a potentiostat configured to measure ionic current through said nanopipette structure versus potential at various potentials in an electrochemical cell containing said nanopipette structure and a reference electrode;
(b) inserting said nanopipette structure into a living cell in said electrochemical cell; and
(c) using said circuit to measure said ionic current, wherein said current is correlated to a known pH.

17. The method of claim 16 wherein said inserting said nanopipette comprises using an SICM and an x-y-z controller.

18. The method of claim 16 or 17 wherein said circuit further comprises an amplifying circuit comprising a detection circuit with gain controls and with a low pass filter for detecting ionic currents.

19. The method of claim 16 or 17 wherein said interior layer comprises a layer of chitosan material having an average pore size between 50 nm and 150 nm diameter.

20. The method of claim 19 wherein the chitosan has a monomer number between about 30,000 and 60,000 units.

21. The method of claim 20 wherein the chitosan comprises a hemeprotein attached thereto.

22. The method of claim 16 wherein the interior layer comprises a polymer coating that is selected from the group consisting of sulfonated tetrafluorethylene copolymer (Nafion®), poly-1-lysine, and alginate.

23. The method of claim 16 wherein the circuit comprises a potentiostat connected to the reference electrode and responsive to input from an amplifier in turn having an input from the working electrode.

24. The method of claim 23 wherein the potentiostat is connected to a counter electrode connected to the reference electrode.

25. The method of claim 23 wherein the working electrode and the counter electrode are Ag/AgCl.

26. The method of claim 23 wherein the voltage is between 0.5V and 0.7V.

27. The method of claim 26 wherein a variety of voltages is set on the potentiostat.

28. The method of claim 23 wherein the pH value is taken on a cancerous cell and compared to a pH on a noncancerous cell.
